(19) United States
(12) Patent Application Publication
(10) Pub. No.: US 2019/0154695 A1
(43) Pub. Date: May 23, 2019

(54) METHOD FOR DETECTING AND ISOLATING INVASIVE CANCER STEM CELLS EMPLOYING CELL-SURFACE AMFR AND THE USE THEREOF

(71) Applicants: Kun-Chih TSAI, Taipei City (TW); Patrick Yuhmin YANG, Hillsborough, CA (US)

(72) Inventors: Kun-Chih TSAI, Taipei City (TW); Patrick Yuhmin YANG, Hillsborough, CA (US); Tze-Sian CHAN, Taipei City (TW)

(21) Appl. No.: 16/314,377

(22) PCT Filed: Jun. 26, 2017
(86) PCT No.: PCT/US17/39319

§ 371 Date: Dec. 28, 2018

Related U.S. Application Data
(60) Provisional application No. 62/355,362, filed on Jun. 28, 2016.

(51) Int. Cl. G01N 33/574; C12N 5/095

(52) U.S. Cl. CPC G01N 33/57492; C12N 5/0695; G01N 33/57415; G01N 33/57446; G01N 33/57426; G01N 33/57434; G01N 33/57438; G01N 33/57423

(57) ABSTRACT

The present disclosure provides an isolated invasive cancer stem cell (iCSC) or a substantially homogeneous iCSC population comprising said iCSC, which is positive for the marker AMFR on the cell membrane. The present disclosure also provides a method of detecting the iCSC within an established cancer or a collection of cancer cells. The present disclosure also provides a method of isolating the iCSC or a substantially homogeneous cell population comprising said iCSC from an established cancer or a collection of cancer cells.

This application claims priority to U.S. Provisional Application Ser. No. 62/355,362 filed Jun. 28, 2016, which is incorporated herein by reference.

This patent application incorporates by reference the material (i.e., Sequence Listing) in the ASCII text file named Sequence_Listing.txt, created on Jun. 28, 2016, having a file size of 5 kilobytes.

### BACKGROUND

### Field of Invention

The present invention relates to a method for detecting and isolating stem cells and the use thereof. More particularly, the present invention relates to a method for detecting and isolating invasive cancer stem cells employing cell-surface AMFR and the use thereof.

### Description of Related Art

A prevailing new paradigm of tumorigenesis proposes that only a subset of tumor cells with stem-like properties, termed “cancer stem cells (CSCs)”, “tumor stem cells”, or “tumor-initiating cells”, has the ability to self-renew and sustain tumorigenesis (Visvader and Lindeman, 2008). Stem-like cancer cells have been found to exist in leukemia, in which they were termed “leukemia stem cells (LSCs)”, and multiple solid tumors, such as glioma, breast cancer, pancreatic cancer, prostate cancer and colon cancer (Ginestier et al., 2007; Lapidot et al., 1994; Li et al., 2007; O'Brien et al., 2007; Singh et al., 2004; van den Hoogen et al., 2010). The discovery of CSCs supports that there is an organizational hierarchy in tumors, which has fundamentally changed our understanding of cancer biology. Importantly, therapies specifically targeting CSCs may shed new lights on the treatment of malignant tumors and hold great promises in improving the outcome of the patients.

The presence of CSCs has profound implications for cancer therapy. At present, all of the phenotypically diverse cancer cells in a tumor are treated as though they have unlimited proliferative potential and can acquire the ability to metastasize. For many years, however, it has been recognized that small numbers of disseminated cancer cells can be detected at sites distant from primary tumors in patients that never manifest metastatic disease. One possibility is that immune surveillance is highly effective at killing disseminated cancer cells before they can form a detectable tumor. Another possibility is that most cancer cells lack the ability to form a new tumor such, that only the dissemination of rare CSCs can lead to metastatic disease. If so, the goal of therapy must be to identify and kill this CSC population.

The identification of CSCs had been facilitated by the discovery of a series of highly specific stem cell or CSC-specific surface markers, which, in conjunction with immunologically labeling techniques and fluorescence assisted cell sorting (FACS), permit the rapid isolation and characterization of CSCs. For instance, in pancreatic ductal adenocarcinoma (PDAC), the cells that co-express the surface markers CD44 and CD133 or CD44, CD24 and epithelial-specific antigen (ESA) have been shown to contain the enriched CSCs (Hermann et al., 2007; Li et al., 2007; Wang et al., 2013). Independently, CSCs in PDAC can be enriched by their high aldehyde dehydrogenase (ALDH) activity, and the presence of ALDH-positive CSCs has been associated with poor prognosis in patients with PDAC (Rasheed et al., 2010). Irrespective of the methods used to isolate CSCs, they carry stem-like cell properties and exhibit increased clonogenic, migratory and invasive potentials in vitro and tumorigenetic potential in vivo. Importantly, tumors originated from CSCs maintain a differentiated phenotype and reproduce the full morphologic and phenotypic heterogeneity of their parental lesions.

A variety sets of cell surface markers have been used to enrich for CSCs from other types of cancers. For instance, gastric cancer cells with the CD90 surface marker had been found to contain the enriched CSCs that possess a high tumorigenetic ability in vivo and self-renewal properties (Jiang et al., 2012). Similarly, the CD90-positive hepatocellular carcinoma cells displayed in vivo tumorigenic capacity and those with CD90 and CD44 demonstrated with a more aggressive phenotype and were prone to distant metastasis in immunodeficient mice (Yang et al., 2008). In human glioma, cells with CD133 were identified to contain to enriched CSCs that are capable of tumor initiation in the brain of immunodeficient mice (Singh et al., 2004). CD133 had also been used as a surface marker for CSCs in lung carcinoma (Eramo et al., 2008), colon cancer (O'Brien et al., 2007) and cholangiocarcinoma (Kokuryo et al., 2012). Of note, a considerable heterogeneity exists with respect to the surface marker that can be used to enrich for CSCs in a specific type of cancer. For instance, recent observation indicated that EpCAM, CD44 and/or CD166, rather than CD133, are more specific marker for CSCs in colon cancer (Dalerba et al., 2007).

Recent evidence suggests that CSCs exist in a dynamic equilibrium with their microenvironments and the CSC phenotype is tightly regulated by both cell-intrinsic and cell-extrinsic factors derived by their surrounding cells or stroma. In keeping with this paradigm, inflammatory cytokines, such as interleukin-6 (IL-6), IL-8, and CCL5, have been found to play an essential role in CSC regulation as well as invasion and metastasis of tumors (Iliopoulos et al., 2011; Korkaya et al., 2012; Korkaya et al., 2011). Among these cytokines, IL-6 is especially important as it has been implicated in the regulation and maintenance of the CSC phenotype in various tumor models and cancer types. The critical role of IL-6 in CSCs may help explain the association between high levels of serum IL-6 levels and a poor prognosis in patients with metastatic breast cancer (Bachelot et al., 2003). At the mechanistical level, it has been shown that the IL-6/Stat3/NF-κB signaling pathway form a positive feedback loop that links inflammation to malignant transformation of mammary epithelial cells (Iliopoulos et al., 2009). IL-6 by itself is sufficient to convert “nonstem” cancer cells to CSCs through paracrinal signaling, thereby maintaining the proportion of CSCs in vivo (Iliopoulos et al., 2011). Constitutive IL-6 expression in breast cancer cells maintains their epithelial-mesenchymal transition (EMT) phenotype, enhances invasiveness, and leads to the formation of poorly differentiated tumors. The EMT phenotype has been implicated in the generation of a CSC phenotype (Mani et al., 2008; Sullivan et al., 2009). In parallel to these findings, a large-scale shRNA screen also identified the essential role of the IL-6/JAK2/Stat3 pathway in the growth and survival of CD44⁺CD24⁻ breast CSCs. Thus, a pharmacological inhibitor of JAK2 could reduce the number of pStat3⁺ cancer cells and tumor growth in a xenograft tumor model (Marotta et al., 2011). Lose of PTEN in a HER2-overexpression genetic background or trastuzumab resistance in breast cancer cells has been linked to activation of an IL-6/Stat3/NF-κB inflammatory loop, which induced an EMT phenotype and expansion of the CSC population. Importantly, a function-blocking anti-IL6 receptor antibody could effectively revert these phenotypes, lending support to its therapeutic potential (Korkaya et al., 2012). Interesting, whereas the IL-6 inflammatory loop induces CSCs with mesenchymal features, another inflammatory cytokine, IL-8, seems to regulate epithelial-like CSCs that express high ALDH activity (Ginestier et al., 2010). This raises the possibility that different cytokines may have distinct roles in maintaining different CSC populations.

There is now a growing body of evidence suggesting that organizational hierarchy exists not only in tumors but also within CSCs. Specifically, different subpopulations of CSCs may differ considerably with each other with respect to their abilities to initiate and maintain tumorigenesis, indicating that there might be considerable phenotypic heterogeneity within CSC subpopulations (Ginestier et al., 2007; Rasheed et al., 2010; Vermeulen et al., 2010; Visvader and Lindeman, 2008). It is now well accepted that therapies specifically targeting this essential subpopulation of cancer cells may shed new lights on the treatment of malignant tumors and hold great promises in improving the outcome of the patients.

A corollary to the CSC model of solid tumorigenesis is that anti-cancer therapies must be directed against CSCs or LSCs to effectively treat solid tumors or hematologic cancers and achieve higher cure rates. Since current therapies are directed against the bulk population, they may be ineffective at eradicating solid tumor stem cells. The limitations of current cancer therapies derive from their inability to effectively kill solid tumor stem cells. The identification of solid tumor stem cells permits the specific targeting of therapeutic agents to this cell population, resulting in more effective cancer treatments. This concept would fundamentally change our approach to cancer treatment.

Invadopodia are transient actin-based protrusions in invasive cancer cells that mediate focal degradation of extracellular matrix (ECM) by the localized proteolytic activity of proteases (Chen, 1989; Paz et al., 2014). Cancer cells use invadopodia during mesenchymal-type migration to degrade and invade ECM structures. Invadopodia are considered as the transformed version of podosomes expressed by motile cells such as macrophages, lymphocytes, dendritic cells, osteoclasts, endothelial cells and smooth muscle cells (Carman et al., 2007; Cougoule et al., 2010; Linder, 2009; Olivier et al., 2006). Podosomes are small (1 μm×0.4 μm in size) and short-lived (minutes) while invadopodia are larger (8 μm×5 μm in size) and can persist for over 1 hour. Structurally, podosomes have a ring-like structure of adhesion-plaque proteins, such as talin, paxillin and vinculin, that surrounds an actin-rich core, whereas invadopodia lack the ring structure and the adhesive protein vinculin in podosomes.

A large number of structural and regulatory proteins participate in the control of actin dynamics during the formation of invadopodia and podosomes. These include the actin regulatory proteins cortactin, Arp2/3, N-WASP, MENA, the adaptor proteins Tks5, Tks4, proteases such as membrane type metalloprotease (MT1-MMP), ADAM12, and fibroblast activation protein (FAP-α), the signaling regulators Src and Arg kinases, and the adhesion molecule β1-integrin (Paz et al., 2014).

Invadopodia or podosomes are organized in response to various signals including cytokines and growth factors, such as TGF-β, TNF-α, SDF-1, and ECM (Schachtner et al., 2013). Multiple signaling transducers are involved in the formation and the maintenance of invadopodia/podosomes, including phospholipase C, protein kinase C (PKC), Src-family tyrosine kinases, and various GTP exchange factors that can then activate Rho GTPases. Src serves as a master swiCSCh for invadopodium or podosome formation by phosphorylating multiple downstream effectors including cortactin, WASP, integrins, paxillin, focal adhesion kinases, Tks5, ASAP1, and p130Cas (Kelley et al., 2010; Schachtner et al., 2013; Soriano et al., 1991; Tarone et al., 1985). Rho GTPases, including Rac, RhoA, and Cdc42, play important roles in invadopodia/podosomes dynamics. Cdc42 and its adaptor protein Nck activates neural Wiskott-Aldrich Symdrome protein (N-WASP), which nucleates actin filaments to initiate invadopodium/podosome formation (Burns et al., 2001; Kelley et al., 2010; Linder et al., 1999; Yamaguchi et al., 2005a). Rac and RhoA contribute to the maturation of invadopodia by phosphorylating and regulating cortactin or cofilin, respectively (Bravo-Cordero et al., 2011; Head et al., 2003). Notably, a proper level and localization of Rho is important for the organization of invadopodia/podosomes as its constitutive expression paradoxically leads to the disruption of podosomes (van Helden and Hordijk, 2011). In osteosarcoma cells, the invadopodia formation is also regulated by the noncanonical Wnt5a-Ror2-Src signaling axis (Enomoto et al., 2009). Moreover, a recent study emphasizes the coordination between the development program epithelial-mesenchymal transition (EMT) and the development of invadopodia. In this regard, the EMT regulator Twist1 induces PDGFRα expression, leading to activation of Src which then phosphorylates the invadopodia components Tks5 and cortactin, leading to invadopodia formation (Eckert et al., 2011). In keeping with the role of EMT in invadopodia formation, it has been shown that TGFβ-induced EMT and invadopodia formation is dependent on Src-mediated phosphorylation of the focal adhesion adaptor Hic-5 (Pignatelli et al., 2012).

Recent research suggests that lipid rafts, a cholesterol-rich specialized membrane microdomain, are required for the assembly and function of invadopodia/podosomes in cancer cells. Specifically, caveolin-1, a resident protein of caveolae, accumulates at Invadopodia and its down-regulation inhibits Invadopodia-mediated ECM degradation (Yamaguchi et al., 2009). Consistently, depletion of caveolin disrupts the association of essential components of invadopodia/podosomes, including Src kinases, β1-integrin and urokinase receptor (uPAR), thereby compromising the migration of cells on ECM (Wei et al., 1999). More in-depth mechanisms underlying the roles of lipid rafts especially caveolae in invadopodia/podosomes await further investigation.

There are now a growing body of evidence revealing that invadopodia exist in vivo and may play a critical role for tumor invasion and metastasis (Gligorijevic et al., 2012; Yamaguchi, 2012; Yamaguchi et al., 2005b). Invadopodia may contribute to cancer cell invasion into the surrounding stroma, intravasation into the vasculature and extravasation (Gligorijevic et al., 2012; Paz et al., 2014). Consistently, intravital imaging revealed invadopodia-like protrusions in tumors cells growing in the mammary fat pad of mice and in tumor cells extending into the blood vessel wall (Gligorijevic et al., 2012; Yamaguchi et al., 2005b). At the functional level, suppressing Invadopodia formation by inhibiting Src, Twist, PDGFRα or Tks5 has been convincingly shown to inhibit tumor metastasis in various tumor models (Eckert et al., 2011).

The autocrine motility factor (AMF) is the secreted form of the glycolytic enzyme glucose-6-phosphate isomerase (GPI), which belongs to the moonlighting family of proteins having multiple functions within a single polypeptide chain. It has been reported that phosphorylation of AMF at serine 185 by casein kinase II facilitates its secretion (Haga et al., 2000). AMF is selectively secreted by tumor cells, but not by normal cells, and stimulates motility and invasiveness of tumor cells and thereby promotes tumor progression and metastasis (Liotta et al., 1986; Nabi et al., 1992; Watanabe et al., 1996). The receptor of AMF, AMF receptor (AMFR), is a 78 kDa cell surface glycoprotein expressed at the plasma membrane particularly concentrating in caveolae and lipid rafts (Benlimame et al., 1998; Le et al., 2000). A growing body of clinical studies indicate that AMFR expression also increases in various malignant tumors and correlates with the metastatic potential of tumors and poor patient survival (Chiu et al., 2008; Hirono et al., 1996; Jiang et al., 2006; Kara et al., 2001; Kawanishi et al., 2000; Kaynak et al., 2005; Maruyama et al., 1995; Nagai et al., 1996; Nakamori et al., 1994; Ohta et al., 2000; Otto et al., 1997; Otto et al., 1994; Takanami et al., 2001; Taniguchi et al., 1998; Tanizaki et al., 2006; Torimura et al., 2001; Wang et al., 2007). Aside from its cell membrane-localized fraction, another fraction of AMFR localizes to the membrane of the endoplasmic reticulum (ER) where it functions as an ubiquitin protein ligase involved in degradation from the ER, an important physiological process termed ER-associated degradation (ERAD) (Fang et al., 2001).

Mechanistic studies have demonstrated that activation of AMFR by AMF can trigger diverse signaling events that together alter the adhesion, motility and proliferation of cancer cells. Stimulation of AMFR with AMF initiates a signaling cascade dependent on protein kinase C, and promotes activation of the Rho-like GTPases, RhoA and Rac1 as well as c-Jun N-terminal kinases (Kanbe et al., 1994; Timar et al., 1999; Torimura et al., 2001; Tsutsumi et al., 2002; Wang et al., 2010; Yanagawa et al., 2004). Activation of these factors induces reorganization of focal contacts and stress fiber formation, leading to enhanced cell motility and proliferation (Silletti et al., 1996). AMF stimulates the motility and MMP-2 secretion of hepatoma cells by up-regulating activated integrin β1 subunit expression (Torimura et al., 2001). A recent study showed that the AMFR-dependent degradation of a metastasis suppressor, KAI1, in the ER contributes to the tumor metastasis in sarcoma (Tsai et al., 2007). AMF can also promote cell motility by activating the β-catenin/Wnt signaling and the activating protein 1 (AP-1) transcription (Kho et al., 2014). Aside from its effects on the migration of tumor cells, AMF also stimulates endothelial cell migration and promotes angiogenesis (Funasaka et al., 2001). Interestingly, some of the activities of AMF is independent of AMFR as it has been shown that AMF directly binds to HER2 and promote its cleavage, thereby activating phophoinositide-3-kiase and mitogen-activated protein kinase signaling in breast cancer cells (Kho et al., 2013).

One of the notable biological functions of AMF is the induction of an epithelial-to-mesenchymal transition (EMT), a developmental program which enables cancer cell dissemination and tumor metastasis. Published studies demonstrated that overexpression of AMF or AMFR induces EMT by enhancing NF-kB transcriptional activity, up-regulating the EMT regulators Snail, ZEB1, ZEB2, and down-regulating the EMT inhibiting micro-RNA miR-200s in various types of cancers, thereby enhancing cell motility in vitro and promoting tumorigenicity and metastasis in vivo (Ahmad et al., 2011; Funasaka et al., 2009; Niinaka et al., 2010; Tsutsumi et al., 2004; Wang et al., 2010). Conversely, silencing of endogenous AMF resulted in mesenchymal-to-epithelial transition with reduced malignancy (Ahmad et al., 2011; Niinaka et al., 2010). The clinical significance of the above findings has been credentialed by an inverse correlation between E-cadherin and AMFR expressions in clinical samples of gastric and bladder cancers (Kawanishi et al., 2000; Otto et al., 1997; Otto et al., 1994).

Once bound to AMFR, the AMF-AMFR complex is internalized via a PI3K-, dynamin- and Rac1-dependent and clathrin-independent raft endocytic pathway to the smooth ER (Benlimame et al., 1998; Le et al., 2002). Interestingly, AMFR has been reported to stably localized to the lipid raft caveolae and partially colocalize with its constituent protein caveolin-1 (Benlimame et al., 1998). As such, caveolin-1 can serve as a negative regulator of the raft-dependent uptake of AMFR (Le et al., 2002). The AMF-AMFR complex can be also internalized via a clathrin-dependent pathway to a specialized endocytic compartment termed multivescular body (Le et al., 2000). Interestingly, the endocytosed AMF and AMFR can be recycled to the site of deposition of fibronection, which may contribute to cellular attachment and the remodeling of the extracellular matrix during cell movement.

### SUMMARY

The present invention relates an invasive cancer stem cell (iCSC) that has the properties of stem cells and are highly invasive or a substantively homogeneous cell population including said iCSC. In particular, the present invention provides the detection or the isolation of said iCSC from an established solid tumor based on the expression of a cell-surface marker AMF. In particular, the present invention provides the detection of said invasive CSCs based on the expression of the molecule AMFR on the surface of invadopodia or podosome-like structures in said iCSC. The present invention also provides a method of screening for pharmaceuticals using said iCSC. The present invention also provides a method or a kit to determine a diagnosis of aggressive solid tumor by employing cell-surface AMFR as a biomarker, and an inhibiting agent of said iCSC.

The present invention also relates an invasive leukemia stem cell (iLSC) that has the properties of stem cells and are highly invasive or a substantively homogeneous cell population including said iLSC. In particular, the present invention provides the detection or the isolation of said iLSC from an established hematopoietic cancer based on the expression of a cell-surface marker AMFR. In particular, the present invention provides the detection of said iLSC based on the expression of the molecule AMFR on the surface of invadopodia or podosome-like structures in said iLSC. The present invention also provides a method of screening for pharmaceuticals using said iLSC. The present invention also provides a method or a kit to determine a diagnosis of aggressive solid tumor by employing cell-surface AMFR as a biomarker, and an inhibiting agent of said iLSC.

Disclosed methods involve obtaining tumor tissues or cells of said solid tumor, contacting said tumor tissues or cells with an effective binding agent, including such as an antibody, a peptide, an aptamer, and a compound, that is capable of binding to AMFR on the cell surface with high affinity, and then determining whether said tumor tissues or cells contains cells that express AMFR on the cell surface.

Disclosed methods also involve obtaining cancer cells of said hematopoietic cancer, contacting said cancer cells with an effective binding agent, including such as an antibody, a peptide, an aptamer, and a compound, that is capable of binding to AMFR on the cell surface with high affinity, and then determining whether said cancer cells contains cells that express AMFR on the cell surface.

In a specific embodiment of the above methods, said binding agent binds to AMFR which is localized to the surface of the invadopodia or podosome-like structures on the cell membrane of said iCSC or iLSC.

In a specific embodiment of the above methods, said malignant solid tumor comprises, but not limited to, pancreatic cancer, lung cancer, liver cancer, glioma, gastric cancer, prostate cancer pancreatic cancer, lung cancer, cholangiocarcinoma, colorectal cancer, and breast cancer.

In another specific embodiment, said CSCs are characterized by: (a) expressing stem cell markers, which comprise, but not limited to, CD133, CD44, CD24, CD90, CD166, epithelial specific antigen (ESA), chemokine (C-X-C motif) receptor 4 (CXCR-4), aldehyde dehydrogenase (ALDH) or any combination of the foregoing; (b) not expressing CD24 if said solid tumor is a breast cancer; (c) giving rise to additional stem-cell-like tumor cells; (d) being able to form a detectable tumor upon transplantation into an immunocompromised host; and/or (e) being able to regenerate the hierarchical organization of solid tumor tissues.

In yet another embodiment, said hematopoietic cancer comprises, but not limited to, acute myeloid leukemia, chronic myeloid leukemia, acute lymphocytic leukemia and chronic lymphocytic leukemia.

In yet another embodiment, said LSCs are characterized by: (a) expressing hematopoietic stem cell markers comprising, but not limited to, CD34, ALDH or both; (b) not expressing CD38; (c) being able to give rise to additional hematopoietic-stem-cell-like cancer cells; (d) being able to form a detectable hematopoietic cancer upon transplantation into an immunocompromised host; and/or (e) being able to regenerate the hierarchical organization of hematopoietic cancers.

In a preferred embodiment, said determining whether tumor tissues or cells contains cells that express AMFR on the cell surface comprise the methods of immunofluorescence staining, immunohistochemistry, immunoblotting, proximity ligation analysis, and/or fluorescence activated cell sorting (FACS).

Disclosed methods involve contacting said tumor cells or cancer cells in said solid tumor or hematopoietic cancer with a binding agent that binds AMFR on the cell surface especially near invadopodia or podosomes-like structures, and them isolating said tumor cells or cancer cells expressing AMFR on the cell surface using FACS, magnetic-assisted cell sorting, or other means that are capable of selecting cells based on specific protein epitopes on the surface.

Disclosed method also involves isolating an iCSC or a substantially homogeneous cell population comprising said iCSC from an established solid tumor, wherein the procedures comprise the steps of: (a) preparing a sample of said solid tumor; (b) contacting said sample with a binding agent that binds to AMFR on the cell surface; and (c) isolating tumor cells from said sample that express AMFR on the cell surface, thereby isolating said iCSC.

Disclosed method involves screening for a pharmaceutical agent, which comprise the steps of: (a) preparing a substantially homogeneous cell population comprising iCSC or iLSC; (b) treating said cell population or an iCSC or an iLSC in said cell population with a test substance; and (c) detecting a change in a biological property of said cell population or said iCSC or said iLSC treated with the test substance, wherein a change in the biological property of said cell population or said iCSC or said iLSC identifies the test substance as the pharmaceutical agent.

Disclosed method additionally involves screening for a pharmaceutical agent, which comprise the steps of: (a) preparing a substantially homogeneous cell population comprising iCSC or iLSC; (b) administrating a test substance and said cell population or said iCSC or said iLSC comprised in said cell population to a non-human animal, (c) detecting tumor formation in said non-human animal, thereby identifying the test substance as the pharmaceutical agent.

Disclosed method also involves isolating an iLSC or a substantially homogeneous cell population comprising said iLSC from an established hematopoietic cancer, wherein the procedures comprise the steps of: (a) preparing a sample of said hematopoietic cancer; (b) contacting said sample with a binding agent that binds to AMFR on the cell surface; and (c) isolating tumor cells from said sample that express AMFR on the cell surface, thereby isolating said iLSC.

The present invention provides a method or a kit for diagnosing an aggressive solid tumor in an individual, which are associated with high likelihoods of invading into surrounding tissues and/or developing metastatic lesions at distant sites.

Disclosed method or kit involves obtaining a first biological sample containing tumor cells from a first individual, determining the frequency of said tumor cells with AMFR on their cell surface in said first biological sample, comparing said frequency in said first biological sample with a second biological sample selected from the group consisting of an earlier obtained biological sample from said individual or a control biological sample obtained from a second individual without said solid tumor; and then determining if said frequency in said first biological sample is different (higher or lower) than that in said second biological sample.

The present invention also provides a method or a kit for diagnosing an aggressive hematopoietic cancer in an individual, which are associated with are associated with high likelihoods of causing severe damage to the bone marrow and/or invading the liver, the lymph nodes, the central nervous system or any tissues outside the bone marrow.

Disclosed method or kit involves obtaining a first biological sample containing cancer cells from said first individual, determining the frequency of said cancer cells with AMFR on their cell surface in said first biological sample, comparing said frequency in said first biological sample with a second biological sample selected from the group consisting of an earlier obtained biological sample from said individual or a control biological sample obtained from a second individual without said hematopoietic cancer, and then determining if said frequency in said first biological sample is different (higher or lower) than that in said second biological sample.

### BRIEF DESCRIPTION OF THE DRAWINGS

FIG. 1A-1C include several panels relating to the expression of AMFR on the surface of a subpopulation of CSCs in primary gastric cancer-derived AGS cells. FIG. 1A shows representative FACS plot showing patterns of CD90 staining of AGS cells. FIG. 1B shows representative FACS plots showing cell surface AMFR staining of CD90⁺ (representing CSCs) and CD90⁻ AGS cells (representing non-stem-like cancer cells; “NSCCs”) with the frequency of the AMFR-positive cell population as a percentage of the respective subgroup of cancer cells shown. FIG. 1C shows the percentages of AMFR-positive cells in CD90⁺ and CD90⁻ AGS cells. ***, P<0.001 versus CD90⁻ cells.

FIG. 2A-2C include several panels relating to the expression of AMFR on the surface of a subpopulation of CSCs in metastatic gastric cancer-derived SNU-16 cells. FIG. 2A shows representative FACS plot showing patterns of CD90 staining of SNU-16 cells. FIG. 2B shows representative FACS plots showing cell surface AMFR staining of CD90⁺ (representing CSCs) and CD90⁻ SNU-16 cells (representing NSCCs), with the frequency of the AMFR-positive cells as a percentage of the respective subgroup of cancer cells shown. FIG. 2C shows the percentages of AMFR-positive cells in CD90⁺ and CD90⁻ SNU-16 cells. ***, P<0.001 versus CD90⁻ cells.

FIG. 3A-3C include several panels relating to the expression of AMFR on the surface of a subpopulation of CSCs in primary pancreatic cancer-derived PANC-1 cells. FIG. 3A shows representative FACS plot showing patterns of CD133 and CD44 staining of PANC-1 cells with the frequency of the boxed CD44⁺CD133⁺ cell population (representing CSCs) as a percentage of cancer cells shown. FIG. 3B shows representative FACS plots showing AMFR staining of CD44⁺CD133⁺ PANC-1 CSCs and cells in the other subpopulations (representing NSCCs) with the frequency of the AMFR-positive cells as a percentage of the respective subgroup of cancer cells shown. FIG. 3C shows the percentages of AMFR-positive cells in CD44⁺CD133⁺ PANC-1 cells and the cells in the other subpopulation. ***, P<0.001 versus CD44⁺CD133⁺ cells.

FIG. 4A-4C include several panels relating to the expression of AMFR on the surface of a subpopulation of CSCs in metastatic pancreatic cancer-derived AsPC-1 cells. FIG. 4A shows representative FACS plot showing patterns of CD133 and CD44 staining of AsPC-1 cells with the frequency of the boxed CD44⁺CD133⁺ cell population (representing CSCs) as a percentage of cancer cells shown. FIG. 4B shows representative FACS plots showing AMFR staining of CD44⁺D133⁺ AsPC-1 CSCs and cells in the other subpopulations (representing NSCCs) with the frequency of the AMFR-positive cells as a percentage of the respective subgroup of cancer cells shown. FIG. 4C shows the percentages of AMFR-positive cell subpopulation in CD44⁺CD133⁺ AsPC-1 cells and the cells in the other subpopulations. ***, P<0.001 versus cells in the other subpopulations.

FIG. 5A-5C includes several panels relating to the expression of AMFR on the surface of a subpopulation of glioma stem cells in malignant glioma-derived U-87MG cells. FIG. 5A shows representative FACS plot showing patterns of CD133 staining of U-87MG cells. FIG. 5B shows representative FACS plots showing surface AMFR staining of CD133⁺ (representing glioma stem cells) or CD133⁻ U-87MG cells (representing NSCCs), with the frequency of the AMFR-positive cells as a percentage of the respective subgroup of glioma cells shown. FIG. 5C shows the percentages of AMFR-positive cell subpopulation in CD133⁺ U-87MG glioma stem cells and those in CD133⁻ cells. ***, P<0.001 versus CD133⁻ cells.

FIG. 6A-6C include several panels relating to the expression of AMFR on the surface of a subpopulation of glioma stem cells in malignant glioma-derived Hs-683 cells. FIG. 6A shows representative FACS plot showing patterns of CD133 staining of Hs-683 cells. FIG. 6B shows representative FACS plots showing cell-surface AMFR staining of CD133⁺ (representing glioma stem cells) or CD133⁻ Hs-683 cells (representing NSCCs), with the frequency of the AMFR-positive cells as a percentage of the respective subpopulation of glioma cells shown. FIG. 6C shows the percentages of AMFR-positive cell subpopulation in CD133⁺ Hs-683 cells and those in CD133⁻ cells. *, P<0.05 versus CD133⁻ cells.

FIG. 7A-7C include several panels relating to the expression of AMFR on the surface of a subpopulation of CSCs in lung adenocarcinoma-derived A-549 cells. FIG. 7A shows representative FACS plot showing patterns of CD133 staining of A-549 cells. FIG. 7B shows representative FACS plots showing surface AMFR staining of CD133⁺ (representing CSCs) and CD133⁻ A-549 cells (representing NSCCs), with the frequency of the AMFR-positive cell population as a percentage of the respective subgroup of cancer cells shown. FIG. 7C shows the percentages of AMFR-positive cell subpopulation in CD133⁺ and CD133⁻ A-549 cells. ***, P<0.001 versus CD133⁻ cells.

FIG. 8A-8C include several panels relating to the expression of AMFR on the surface of a subpopulation of CSCs in lung squamous cell carcinoma-derived H-520 cells. FIG. 8A shows representative FACS plot showing patterns of CD133 staining of H-520 cells. FIG. 8B shows representative FACS plots showing surface AMFR staining of CD133⁺ (representing CSCs) and CD133⁻ H-520 cells (representing NSCCs), with the frequency of the AMFR-positive cell population as a percentage of the respective subgroup of cancer cells shown. FIG. 8C shows the percentages of AMFR-positive cell subpopulation in CD133⁺ and CD133⁻ H-520 cells. ***, P<0.001 versus CD133⁻ cells.

FIG. 9A-9C include several panels relating to the expression of AMFR on the surface of a subpopulation of CSCs in primary prostate cancer-derived 22Rv-1 cells. FIG. 9A shows representative FACS plot showing patterns of CD133 and CD44 staining of 22Rv-1 cells with the frequency of the boxed CD44⁺CD133⁺ cell population (representing CSCs) as a percentage of cancer cells shown. FIG. 9B shows representative FACS plots showing surface AMFR staining of CD133⁺CD44⁺ 22Rv-1 CSCs and cells in the other subpopulations (representing NSCCs), with the frequency of the AMFR-positive cell population as a percentage of the respective subgroup of cancer cells shown. FIG. 9C shows the percentages of AMFR-positive cell subpopulation in CD133⁺CD44⁺ 22Rv-1 CSCs and the cells in the other subpopulation. ***, P<0.001 versus cells in the other subpopulations.

FIG. 10A-10C include several panels relating to the expression of AMFR on the surface of a subpopulation of CSCs in metastatic prostate cancer-derived PC-3 cells. FIG. 10A shows representative FACS plot showing patterns of CD133 and CD44 staining of PC-3 cells with the frequency of the boxed CD44⁺CD133⁺ cell population (representing CSCs) as a percentage of cancer cells shown. FIG. 10B shows representative FACS plots showing surface AMFR staining of CD133⁺CD44⁺ PC-3 CSCs and cells in the other subpopulations (representing NSCCs), with the frequency of the AMFR-positive cell population as a percentage of the respective subgroup of cancer cells shown. FIG. 10C shows the percentages of AMFR-positive cell subpopulation in CD133⁺CD44⁺ PC-3 CSCs and those in the other subpopulation. **, P<0.01 versus cells in the other subpopulations.

FIG. 11A-11C include several panels relating to the expression of AMFR on the surface of the majority of LSCs in acute myeloid (monocytic) leukemia-derived THP-1 cells. FIG. 11A shows representative FACS plot showing patterns of CD34 and CD38 staining of THP-1 cells with the frequency of the boxed CD34⁺CD38⁻ cell population (representing LSCs) as a percentage of leukemia cells shown. FIG. 11B shows representative FACS plots showing surface AMFR staining of CD34⁺CD38⁻ THP-1 LSCs and cells in the other subpopulations, with the frequency of the AMFR-positive cell population as a percentage of the respective subgroup of leukemia cells shown. FIG. 11C shows the percentages of AMFR-positive cell subpopulation in CD34⁺CD38⁻ THP-1 LSCs and the cells in the other subpopulation. ***, P<0.001 versus cells in the other subpopulations.

FIG. 12A-12C include several panels relating to the expression of AMFR on the surface of the majority of LSCs in acute myeloid (promyeloblast) leukemia-derived HL-60 cells. FIG. 12A shows representative FACS plot showing patterns of CD34 and CD38 staining of HL-60 cells with the frequency of the boxed CD34⁺CD38⁻ cell population (representing LSCs) as a percentage of leukemia cells shown. FIG. 12B shows representative FACS plots showing surface AMFR staining of CD34⁺CD38⁻ HL-60 LSCs and cells in the other subpopulations, with the frequency of the AMFR-positive cell population as a percentage of the respective subgroup of leukemia cells shown. FIG. 12C shows the percentages of AMFR-positive cell subpopulation in CD34⁺CD38⁻ HL-60 LSCs and the cells in the other subpopulations. ***, P<0.001 versus cells in the other subpopulations.

FIG. 13A-13B include several panels relating to the expression of EMT- and pluripotency-associated markers in AMFR-positive and AMFR-negative CSCs and NSCCs. FIG. 13A shows the relative transcript levels of the EMT-associated genes CDH2, FOXC2, FN1, SNAI2, TWIST1, VIM, ZEB1 and ZEB2 in CD133⁺CD44⁺AMFR⁺ pancreatic cancer PANC-1 cells (representing AMFR-positive CSCs), CD133⁺CD44⁺AMFR⁻ cells (representing AMFR-negative CSCs), and cells in the other populations cells (representing NSCCs) using qRT-PCR analysis. FIG. 13B shows the relative transcript levels of the pluripotency- or sternness-associated genes ALDH, THY1, MYC, OCT4, IL6, and IL8 in CD133⁺CD44⁺AMFR⁺ PANC-1 cells, CD133⁺CD44⁺AMFR⁻ cells, and cells in the other populations cells using qRT-PCR analysis. Data are represented as mean±SEM; n=3. *P<0.05 vs. cells in the other subpopulations; †P<0.05 vs. CD44⁺CD133⁺AMFR⁻ cells.

FIG. 14A-14B include several panels relating to the strong invasive property of AMFR-positive CSCs. The invasive capacities of CD133⁺CD44⁺AMFR⁺ pancreatic cancer PANC-1 cells (representing AMFR-positive CSCs), CD44⁺CD133⁺AMFR⁻ cells (representing AMFR-negative CSCs), and cells in the other populations (representing NSCCs) in response to pancreatic stromal stellate cell-conditioned media in a modified Boyden chamber assay. Shown in FIG. 14A are representative immunofluorescence images of the invaded cells, with cell nuclei stained with CYTOX-green (green). Scale bars=100 μm. FIG. 14B shows the numbers of invaded cells in FIG. 14A. Data are represented as mean±SEM; n=3. *P<0.05 vs. cells in the other subpopulations; †P<0.05 vs. CD44⁺CD133⁺AMFR⁻ cells.

FIG. 15A-15B include several panels relating to the invadopodia formation in AMFR-positive and AMFR-negative CSCs and NSCCs. FIG. 15A shows confocal views of CD44⁺CD133⁺AMFR⁺ pancreatic cancer PANC-1 cells (representing AMFR-positive CSCs), CD44⁺CD133⁺AMFR⁻ cells (representing AMFR-negative CSCs) and the cells in the other subpopulations (representing NSCCs) showing invadopodia (yellow dots) with the colocalized invadopodia markers cortactin (green) and F-actin (red) that penetrate into the underlying HDFC matrix. Scale, 10 μm. FIG. 15B shows quantification of invadopodia in FIG. 15A. Data are represented as mean±SEM; n=3. *P<0.05 vs. cells in the other subpopulations; †P<0.05 vs. CD44⁺CD133⁺AMFR⁻ cells.

FIG. 16A-16B include several panels relating to the localization of AMFR to the invadopodium in CSCs. FIG. 16A shows confocal views of CD44⁺CD133⁺AMFR⁺ PANC-1 cells (representing AMFR-positive CSCs), CD44⁺CD133⁺AMFR⁻ cells (representing AMFR-negative CSCs) and the cells in the other subpopulations (representing NSCCs) showing the invadopodia structures with colocalized (yellow dots; merge) AMFR (red) and the invadopodia marker cortactin (green). FIG. 16B shows quantification of the staining in invadopodia in FIG. 16A. Data are represented as mean±SEM; n=3. *P<0.05 vs. cells in the other subpopulations; †P<0.05 vs. CD44⁺CD133⁺AMFR⁻ cells.

FIG. 17A-17B include several panels relating to the detection of invadopodia proteins and the presence of AMFR in the invadopodia of CSCs. FIG. 17A shows representative immunoblots of cortactin, TKS-5, actin, caveolin-1 and β1-integrin in the invadopodia and cell body fractions isolated from pancreatic cancer PANC-1 cells seeded on HDFC matrices. FIG. 17B shows representative immunoblots of AMFR, cortactin, TKS-5, and β-actin in the invadopodia fraction of CD133⁺CD44⁺ PANC-1 cells (representing CSCs) and the cells in the other subpopulations (representing NSCCs). Note the AMFR is predominantly present in the invadopodia of CD133⁺CD44⁺ CSCs.

FIG. 18A-18B include several panels relating to the localization of AMFR in the invadopodia/podosome of LSCs. FIG. 18A shows confocal views of phorbal 12-myristate 13-acetate-treated CD34⁺CD38⁻ THP-1 cells showing invadopodia- or podosome-like structures with colocalized (yellow dots; merge) AMFR (red) and the invadopodia marker cortactin (green). Cells in the other subpopulations (representing non-LSCs) were treated and stained as controls. FIG. 18B shows quantification of the staining in invadopodia in FIG. 18A. Data are represented as mean±SEM; n=3. ***P<0.001 vs. cells in the other populations.

FIG. 19A-19B include several panels relating to the growth and the dissemination patterns of AMFR-positive or AMFR-negative CSCs in an orthotopic model of pancreatic tumor progression. FIG. 19A shows representative BLI of the tumors generated by CD133⁺CD44⁺AMFR⁺ (representing AMFR-positive CSCs) and CD133⁺CD44⁺AMFR⁻ PANC-1 cells (representing AMFR-negative CSCs) at 6 weeks following cell inoculation. FIG. 19B shows tumor bulk in FIG. 19A quantified as BLI normalized photon counts as a function of time. Data are represented as mean±SEM; n=6. P<0.01; ***P<0.001 vs. CD133⁺CD44⁺AMFR⁻ cells.

### DETAILED DESCRIPTION

It is well documented that many types of tumors contain cancer cells with heterogeneous phenotypes, reflecting aspects of the differentiation that normally occurs in the tissues from which the tumors arise. The variable expression of normal differentiation markers by cancer cells in a tumor suggests that some of the heterogeneity in tumors arises as a result of the anomalous differentiation of tumor cells. Examples of this include the variable expression of myeloid markers in chronic myeloid leukemia, the variable expression of neuronal markers within peripheral neurectodermal tumors, and the variable expression of milk proteins or the estrogen receptor within breast cancer.

Acute myeloid leukemia (AML) is characterized by the clonal expansion of immature myeloblasts initiating from rare LSCs. Studies have demonstrated that a certain ratio of leukemia consists of a heterogeneous cell fraction and is not configured with a homogenous cell population capable of clonal proliferation. Lapidot and Dick identified such heterogeneity in AML and reported that CD34+CD38− cells are transplanted selectively in CB17-scid and NOD/SCID mice (Lapidot et al., 1994). LSCs are responsible for tumor maintenance, and also give rise to large numbers of abnormally differentiating progeny that are not tumorigenic, thus meeting the criteria of cancer stem cells. Tumorigenic potential is contained within a subpopulation of cancer cells differentially expressing the markers of the present invention.

Recent studies have shown that, similar to leukemia and other hematologic malignancies, tumorigenic and non-tumorigenic populations of solid tumor cells, such as breast cancer cells, can be isolated based on their expression of cell surface markers (Al-Hajj et al., 2003; Ginestier et al., 2007). In many cases of solid tumor, only a small subpopulation of cells termed CSCs, tumor stem cells, or tumor-initiating cells had the ability to form new tumors. This work strongly supports the existence of CSCs in breast cancer. Further evidence for the existence of cancer stem cells occurring in solid tumors has been found in central nervous system (CNS) malignancies (Singh et al., 2004). Using culture techniques similar to those used to culture normal neuronal stem cells it has been shown that neuronal CNS malignancies contain a small population of cancer cells that are clonogenic in vitro and initiate tumors in vivo, while the remaining cells in the tumor do not have these properties.

CSCs in solid tumors are functionally characterized by being tumorigenetic, being able to give rise to additional tumorigenic cells (“self-renew”) and non-tumorigenic tumor cells (“differentiation”). The origins of solid tumor stem cells vary between different types of cancers or solid malignant tumors. Solid tumor stem cells may arise either as a result of genetic damage that deregulates the proliferation and differentiation of normal stem cells (Lapidot et al., Nature 367(6464): 645-8 (1994)) or by the dysregulated proliferation of a normal restricted progenitor or a normal differentiated cell type. Typically, solid tumors are visualized and initially identified according to their locations, not by their developmental origin.

The isolation and characterization of CSCs or LSCs have borrowed the concepts and principles of normal stem cell biology. For instance, CSCs and LSCs can be operationally characterized by cell surface markers recognized by reagents that specifically bind to the cell surface markers. It has often been possible to identify combinations of positive and negative markers that uniquely identify stem cells and allow their substantial enrichment in other contexts (see Morrison et al., Cell 96(5): 737-49 (1999); Morrison et al., Proc. Natl. Acad. Sci. USA 92(22): 10302-6 (1995); Morrison & Weissman, Immunity 1(8): 661-73 (1994)). For example, proteins, carbohydrates, or lipids on the surfaces of solid tumor stem cells can be immunologically recognized by antibodies specific for the particular protein or carbohydrate (for construction and use of antibodies to markers, see, Harlow, Using Antibodies: A Laboratory Manual (Cold Spring Harbor Press, Cold Spring Harbor, N.Y., 1999). The set of markers present on the cell surfaces of solid tumor stem cells (the “cancer stem cells” of the invention) and absent from the cell surfaces of these cells is characteristic for solid tumor stem cells. Therefore, solid tumor stem cells can be selected by positive and negative selection of cell surface markers. A reagent that binds to a solid tumor stem cell is a “positive marker” (i.e. a marker present on the cell surfaces of solid tumor stem cells) that can be used for the positive selection of solid tumor stem cells. A reagent that binds to a solid tumor stem cell “negative marker” (i.e., a marker not present on the cell surfaces of solid tumor stem cells but present on the surfaces of other cells obtained from solid tumors) can be used for the elimination of those solid tumor cells in the population that are not solid tumor stem cells (i.e., for the elimination of cells that are not solid tumor stem cells).

Except cell surface markers, solid tumor stem cells can be operationally characterized by enzymatic markers. In a particular embodiment, said solid tumor stem cells can be characterized by the expression or the enzymatic activity of aldehyde dehydrogenase 1 (ALDH1). For example, the ALDH positive cell population, representing 6% of the normal breast epithelial cells, has stem cell characteristics. Phenotypic markers associated with stem and progenitor cells segregated with the ALDH positive population. Also the mammosphere initiating cells, which according to previous studies are likely to be the normal breast stem cells are contained in the ALDH positive fraction of the mammary epithelium (see U.S. Pat. No. 8,435,746). Furthermore, the ALDH positive population contains the cancer stem cell population, as shown by the ability to generate tumors in mice. As few as 500 ALDH positive cells generate tumors upon implantation in NOD/SCID mice, whereas the ALDH negative population is not tumorigenic, even when implanted in high numbers (50,000). The latency and size of the tumor correlated with the number of ALDH+ cell implanted.

ALDH positive cells can be detected in situ by immunostaining with ALDH 1 antibody or by the FACS-based enzymatic assay.

In order to access the proliferation or “tumor-initiating potential of CSCs in vivo, CSCs can be injected into animals, preferably mammals, more preferably in rodents such as mice, and most preferably into immunocompromised mice, such as SCID mice, Beige/SCID mice or NOD/SCID mice. NOD/SCID mice are injected with the varying number of cells and observed for tumor formation. The injection can be by any method known in the art, following the enrichment of the injected population of cells for solid tumor stem cells.

In order the access the proliferative potential of CSCs in vitro, CSCs can be obtained from solid tumor tissue by dissociation of individual cells. Tissue from a particular tumor is removed using a sterile procedure, and the cells are dissociated using any method known in the art (see, Sambrook et al., Molecular Cloning: A Laboratory Manual (Cold Spring Harbor Press, Cold Spring Harbor, N.Y., 1989); Current Protocols in Molecular Biology, Ausubel et al., eds., (Wiley Interscience, New York, 1993), and Molecular Biology LabFax, Brown, ed. (Academic Press, 1991)), including treatment with enzymes such as trypsin, collagenase and the like, or by using physical methods of dissociation such as with a blunt instrument. Methods of dissociation are optimized by testing different concentrations of enzymes and for different periods of time, to maximize cell viability, retention of cell surface markers, and the ability to survive in culture (Worthington Enzyme Manual, Von Worthington, ed., Worthington Biochemical Corporation, 2000). Dissociated cells are centrifuged at low speed, between 200 and 2000 rpm, usually about 1000 rpm (210 g), and then resuspended in culture medium. For guidance to methods for cell culture, see Spector et al., Cells: A Laboratory Manual (Cold Spring Harbor Press, Cold Spring Harbor, N.Y., 1998). The dissociated tumor cells can be placed into any known culture medium capable of supporting cell growth, including HEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. However, a preferred embodiment for proliferation of solid tumor stem cells is to use a defined, low-serum culture medium. A preferred culture medium for solid tumor stem cells is a defined culture medium comprising a mixture of Ham's F12, 2% fetal calf serum, and a defined hormone and salt mixture, either insulin, transferrin, and selenium or B27 supplement. Brewer et al., J. Neuroscience Res. 35: 567 (1993).

In certain embodiments, in vitro proliferation of CSCs isolated from pancreatic cancer is assessed by placing cells in serum-free medium, such as the Neurobasal Media (Invitrogen/Life Technologies) containing Glutamax, bFGF (20 ng/ml), EGF (20 ng/ml), N-2 and B27, at 10,000 cells/well in multi-well ultra-low attachment plates (Corning, Lowell, Mass., USA) (see Arensman et al. Oncogene 33: 899-908 (2014)). Likewise, in vitro proliferation of gastric cancer stem cells is assessed by placing cells in serum-free medium, such as epithelial basal medium (EBM-2; Lonza), supplemented with 4 mg/ml insulin (Sigma-Aldrich), B27, 20 ng/ml EGF and 20 ng/ml basic fibroblast growth factor (Invitrogen), in ultra-low attachment plates (see Jiang et al. Oncogene 31: 671-682 (2011)). In vitro proliferation of hepatoma stem cells is assessed by placing cells in serum-free medium, such as DMEM/F12 medium (Invitrogen), supplemented with 20 ng/ml human recombinant EGF (Sigma-Aldrich), 10 ng/ml human recombinant bFGF (Invitrogen), 4 μ/ml insulin (Sigma-Aldrich), B27 (1:50; Invitrogen), 500 units/ml penicillin (Invitrogen) and 500 μg/ml streptomycin (Invitrogen), in ultra-low attachment plates (see Ma et al., Cell Stem Cell 7: 694-707 (2010)). In vitro proliferation of glioma stem cells is assessed by placing cells in serum-free medium, such as NSC proliferation medium (StemCell), supplemented with 20 ng/ml EGF (Sigma-Aldrich), 10 ng/ml bFGF (Sigma-Aldrich) and 0.3% agarose (Sigma-Aldrich) (see Zheng et al. Nature 455(23): 1129-133 (2008)). In vitro proliferation of glioma stem cells is assessed by placing cells in serum-free medium, such as DMEM/F12 medium, supplemented with glucose to 0.6%, 1% penicillin/streptomycin, 2 mM L-glutamine (Invitrogen), 4 μg/ml heparin, 5 mM HEPES, 4 mg/ml BSA (Sigma-Aldrich), 10 ng/ml FGF basic and 20 ng/ml EGF (R&D Systems) (see Dieter et al. Cell Stem Cell 9: 357-65 (2011)). Cells are replenished with supplemented medium every second day. After 7-14 days, tumor spheres were visualized and counted by phase contrast microscopy. To propagate spheres in vitro, spheres were collected by gentle centrifugation and were dissociated to single cells using TrypLE Express (Invitrogen) or accutase (Millipore). Following dissociation, trypsin inhibitor (Invitrogen) was used to neutralize the reaction, and cells were sieved through a 40-μm filter and re-seeded to generate spheres of the next generation.

The above-mentioned methods of accessing the proliferative potential of iCSCs or iLSCs in vitro can be employed for expanding the above mentioned iCSCs or iLSCs or substantially homogenous cell populations comprising said iCSCs or iLSCs.

Non-human animals, immunodeficient animals can be used for the grafting of the present invention since they are unlikely to have rejection reactions. Immunodeficient animals preferably used include non-human animals that lack functional T cells, for example, nude mice and nude rats, and non-human animals that lack both functional T and B cells, for example, SCID mice and NOD-SCID mice. It is more preferably to use mice that lack T, B, and NK cells and have excellent transplantability, including, for example, NOG or NSG mice. Regarding the weekly age of non-human animals, for example, 4 to 100-week-old athymic nude mice, SCID mice, NOD-SCID mice, or NOG mice are preferably used. NOG mice can be prepared, for example, by the method described in WO 2002/043477, and are available from the Central Institute for Experimental Animals or the Jackson Laboratory (NSG mice).

Cells to be grafted may be any cells, including cell masses, tissue fragments, individually dispersed cells, cells cultured after isolation, and cells isolated from a different animal into which the cells have been grafted; however, dispersed cells are preferred. The number of grafted cells may be 10.sup.6 or less; however, it is acceptable to graft more cells.

With respect to the grafting site, subcutaneous grafting is preferred because the graft technique is simple. The grafting site is not particularly limited, and it is preferable to select an appropriate grafting site depending on the animal used. There is no particular limitation on the grafting operation of NOG-established cancer cell lines, and the cells can be grafted by conventional grafting operations.

Detection and Isolation of AMFR-Positive CSC or LSC

The current invention provides methods for detecting iCSC or iLSC or substantially homogeneous iCSC or iLSC populations by determining the expression of AMFR on the cell surface in a biological sample obtained from an individual with a solid tumor or a hematopoietic cancer.

The human AMFR (NCBI Entrez Gene 267) encodes a glycosylated transmembrane receptor. Its ligand, autocrine motility factor, is a tumor motility-stimulating protein secreted by tumor cells. The encoded receptor is also a member of the E3 ubiquitin ligase family of proteins. It catalyzes ubiquitination and endoplasmic reticulum-associated degradation of specific proteins. AMFR is located on chromosome 16 at gene map locus 16q21 and molecular mass of 72.996 Kd. AMFR sequences are publically available, for example form NCBI GenBank (e.g., accession numbers NM_001144.5 (mRNAs) and NP_001135.3 (proteins)).

In a preferred embodiment, determining the expression of AMFR on the cell surface involve determining the expression of AMFR on the surface of invadopodia of podosomes-like structures in cells. Said invadopodia or podosomes-like structures are transient actin-based protrusions in a tumor cell or a leukemia cell that mediate focal degradation of extracellular matrix and cell invasion and tumor metastasis.

In these methods, first, samples obtained from cancer patients are prepared. In the present invention, a “sample” is not particularly limited as long as it is preferably an organ or tissue derived from a cancer patient. It is possible to use a frozen or unfrozen organ or tissue. Such samples include, for example, cancer (tumor) tissues isolated from cancer patients. In these methods, a sample is then contacted with an AMFR-binding agent.

Specifically, for example, organs or tissues are isolated from cancer patients, and specimens are prepared. The specimens can be used to detect, identify, or quantify the presence of cancer stem cells. Specimens can be appropriately prepared by using known methods, for example, the PFA-AMeX-Paraffin method (WO 09/078,386). The samples include, for example, frozen or unfrozen organs or tissues. First, samples from cancer patients are fixed in a PFA solution. “PFA solution” refers to a cell fixation solution which is an aqueous solution of 1 to 6% paraformaldehyde combined with a buffer such as phosphate buffer. It is preferable to use 4% PFA fixation solution (4% paraformaldehyde/0.01 M PBS (pH7.4)). For fixation with a PFA fixation solution, organs or tissues of interest are immersed in a PFA solution containing 1 to 6%, preferably 4% paraformaldehyde, at 0 to 8 Celsius degree., preferably at about 4 Celsius degree, for 2 to 40 hours, preferably for 6 to 30 hours. Then, fixed organs or tissues are washed with phosphate buffered saline or such. Washing may be carried out after excising portions from the observed organs or tissues.

Organs or tissues thus prepared are then embedded in paraffin by the AMeX method. The AMeX method is a paraffin embedding method with a series of the following steps: cold acetone fixation, dehydration with acetone, clearing in methylbenzoate and xylene, and paraffin embedding. Specifically, tissues are immersed in acetone at −25 to 8 Celsius degree, preferably at −20 to 6 Celsius degree, for 2 to 24 hours, preferably for 4 to 16 hours. Then, the tissues in acetone are warmed to room temperature. Alternatively, organs or tissues are transferred into acetone at room temperature. Then, dehydration is performed for 0.5 to 5 hours, preferably 1 to 4 hours at room temperature. Subsequently, the organs or tissues are cleared by immersion in methylbenzoate at room temperature for 0.5 to 3 hours, preferably for 0.5 to 2 hours, followed by immersion in xylene at room temperature for 0.5 to 3 hours, preferably 0.5 to 2 hours. Next, the organs or tissues are embedded in paraffin by penetration at 55 to 65 Celsius degree, preferably at 58 to 62 Celsius degree for 1 to 4 hours, preferably for 1 to 3 hours. The paraffin blocks of organs or tissues prepared by the PFA-AMeX method are stored at low temperature before use.

At the time of use, the paraffin blocks thus prepared are sliced into thin sections using a microtome or the like. Then, the thin sections are deparaffinized and rehydrated. Deparaffinization and rehydration can be performed by known methods. For example, deparaffinization can be performed using xylene and toluene, while rehydration can be carried out using alcohol and acetone. The resulting thin sections are stained, for example, by histochemistry, immunohistochemistry, or enzyme histochemistry for detection, identification, or quantitation. When the prepared samples are stained by histochemistry (special staining), it is possible to use any staining method commonly available for paraffin-embedded sections (for example, PAS staining, giemsa staining, and toluidine blue staining). For staining by enzyme histochemistry, the sections may be stained by any staining method available for sections (for example, various staining such as with ALP, ACP, TRAP, or esterase). In addition, histopathological tissues can be stained by the following: hematoxylin-eosin staining for general staining; van Gieson staining, azan staining, and Masson Trichrome staining for collagen fiber staining; Weigert staining and Elastica van Gieson staining for elastic fiber staining; Watanabe's silver impregnation staining and PAM staining (periodic acid methenamine silver stain) for reticular fibers/basal membrane staining, etc.

Staining with immunohistochemistry and enzyme histochemistry can be performed by direct methods using primary antibodies labeled with an enzyme or labeling substance, or indirect methods using non-labeled primary antibodies and labeled secondary antibodies. However, such methods are not limited thereto. Antibodies can be labeled by conventional methods. Labeling substances include, for example, radioisotopes, enzymes, fluorescent substances, and biotin/avidin.

The labeling substances may be those commercially available. Radioisotopes include, for example, .sup.32P, .sup.33P, .sup.131I, .sup.125I, .sup.3H, .sup.14C, and .sup.35S. Enzymes include, for example, alkaline phosphatase, horse radish peroxidase, .beta.-galactosidase, and .beta.-glucosidase. Fluorescent substances include, for example, fluorescein isothiocyanate and rhodamine. These may be commercially available. Labeling can be carried out by known methods.

Thin sections are stained, for example, by histochemistry, immunohistochemistry, or enzyme histochemistry for detection, identification, or quantitation.

In exemplary methods, determining the protein expression of AMFR on the cell surface comprises the use of antibodies specific to AMFR on the cell surface and immunohistochemistry staining on fixed (e.g., formalin-fixed) and/or wax-embedded (e.g., paraffin-embedded) pancreatic tumor tissues. Fixatives for tissue preparations or cells are well known in the art and include formalin, gluteraldehyde, methanol, or the like (Carson, Histotechology: A Self-Instructional Text, Chicago: ASCP Press, 1997). The immunohistochemistry methods may be performed manually or in an automated fashion.

Antibody reagents can be used in assays to detect expression of AMFR on the cell surface especially near the invadopodium or podosomes-like structures of tumor or cancer cells in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to AMFR can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FICSC), Oregon Green™ rhodamine, Texas red, tetrarhodimine isothiocynate (TRICSC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

We contemplated kits useful for facilitating the practice of a disclosed method. In one embodiment, kits are provided for detecting AMFR on the surface especially near the invadopodium or podosomes-like structures of said tumor cells or hematopoietic cancer cells from an individual with a malignant solid tumor or a hematopoietic cancer. In a preferred embodiment, a kit is provided for detecting AMFR protein on the cell surface in combination with one to a plurality of housekeeping genes or proteins (e.g., β-actin, GAPDH, RPL13A, tubulin, and the likes well known in the art of protein biochemistry). The detection means can include means for detecting AMFR protein on the cell surface, such as an antibody or antibody fragment specific for the AMRF protein, or an aptamers specific for the AMFR protein. In a particular example, kits can include an antibody specific for the AMFR protein on the cell surface. Particular kit embodiments can further include, for instance, one or more (such as two, three or four) antibodies specific for a selected group of housekeeping proteins.

In some preferred embodiments, the primary detection means (e.g., nucleic acid probe, nucleic acid primers, or antibody) can be directly labeled with a fluorophore, chromophore, or enzyme capable of producing a detectable product (e.g., alkaline phosphates, horseradish peroxidase and others commonly known in the art). In other embodiments, kits are provided including secondary detection means, such as secondary antibodies or non-antibody hapten-binding molecules (e.g., avidin or streptavidin). In some such instances, the secondary detection means will be directly labeled with a detectable moiety. In other instances, the secondary or higher order antibody can be conjugated to a hapten (e.g., biotin, DNP, or FICSC), which is detectable by a cognate hapten binding molecule (e.g., streptavidin horseradish peroxidase, streptavidin alkaline phosphatase, or streptavidin QDot™). Some kit embodiments can include colorimetric reagents in suitable containers to be used in concert with primary, secondary or higher order detection means that are labeled with enzymes for the development of such colorimetric reagents.

Antibodies or aptamers used in the methods provided here can be obtained from a commercially available source or prepared using techniques well known in the art. Antibodies are immunoglobulin molecules (or combinations thereof) that specifically binds to, or is immunologically reactive with, a particular antigen, and includes polyclonal, monoclonal, genetically engineered and otherwise modified forms of antibodies, including but not limited to chimeric antibodies, humanized antibodies, hetero-conjugate antibodies, single chain Fv antibodies, polypeptides that contain at least a portion of an immunoglobulin that is sufficient to confer specific antigen biding to the polypeptide, and antigen binding fragments of antibodies. Antibody fragments include proteolytic antibody fragments, recombinant antibody fragments, complementarity determining region fragments, camelid antibodies (e.g., U.S. Pat. Nos. 6,015,695; 6,005,079; 5,874,541; 5,840,526; 5,800,988; and 5,759,808), and antibodies produced by cartilaginous and bony fishes and isolated binding domains thereof.

TABLE 1 shows exemplary commercial sources of antibodies for AMFR.

TABLE 1
Exemplary commercial sources of AMFR-specific antibodies

| Antibody type | Source | Catalog number | Application |
|---|---|---|---|
| Rabbit Polyclonal | Aviva Systems | ARP42995_T100 | WB |
| | Biology | | |
| Rabbit Polyclonal | Novus | NBP2-15374 | WB, IHC |
| | Biologicals | | |
| Rabbit Polyclonal | LifeSpan | LS-C144946-100 | IHC, WB |
| | BioSciences | | |
| Mouse Monoclonal | Abcam | ab54787 | WB |
| Rabbit Polyclonal | Abcam | ab191163 | WB, IHC |
| Rabbit Polyclonal | United States | 031834-AP-200ul | WB |
| | Biological | | |
| Rabbit Polyclonal | United States | 031834-APC-200 | IF, WB |
| | Biological | ul | |
| Rabbit Polyclonal | Invitrogen | PA5-29501 | WB, IHC |
| | Antibodies | | |
| Rabbit Polyclonal | Invitrogen | PA5-12051 | WB, IHC, FACS |
| | Antibodies | | |
| Rabbit Polyclonal | GeneTex | GTX112393 | IHC-P, WB |
| Rabbit Polyclonal | Novus | NBP1-49986 | WB, IP |
| | Biologicals | | |
| Rabbit Polyclonal | Novus | 35450002 | WB, ELISA, IHC-P |
| | Biologicals | | |
| Rabbit Polyclonal | Abgent | AP2162a | WB, FC, IHC |
| Rabbit Polyclonal | LifeSpan | LS-C319452-200 | ELISA, WB |
| | BioSciences | | |
| Rabbit Polyclonal | LifeSpan | LS-C251260-200 | ELISA, WB |
| | BioSciences | | |
| Rabbit Polyclonal | MyBioSource | MBS8502450 | WB, IF, IHC |
| Rabbit Polyclonal | MyBioSource | MBS9206813 | WB, ELISA |
| Rabbit Polyclonal | Biorbyt | orb163699 | WB |
| Rabbit Polyclonal | Biorbyt | orb181626 | WB, IHC-P |
| Rabbit Polyclonal | Abbexa Ltd | abx025052 | FCM |
| Rabbit Polyclonal | Abbexa Ltd | abx002685 | WB, IHC, IF/ICC |
| Rabbit Polyclonal | NSJ | F40060-0.08ML | WB, IHC, FACS, |
| | Bioreagents | | ELISA |
| Rabbit Polyclonal | Source | SBS503728 | WB, IHC |
| | BioScience | | |
| Rabbit Polyclonal | Source | SBS408404 | IHC, WB |
| | BioScience | | |
| Rabbit Polyclonal | Abnova | PAB5236 | WB, ELISA |
| | Corporation | | |
| Mouse Monoclonal | Abnova | H00000267-M01 | ELISA, WB |
| | Corporation | | |
| Rabbit Polyclonal | Cell Signaling | 9590S | WB, IP |
| | Technology | | |
| Rabbit Polyclonal | Acris | AP50160PU-N | WB |
| | Antibodies | | |
| | GmbH | | |
| Rabbit Polyclonal | Acris | AP12067PU-N | WB, IHC |
| | Antibodies | | |
| | GmbH | | |
| Rabbit Polyclonal | antibodies-online | ABIN2199949 | IHC, WB |
| Mouse Monoclonal | antibodies-online | ABIN2199947 | ELISA, WB |
| Rabbit Polyclonal | St John's | STJ22602 | WB, IHC, IF |
| | Laboratory | | |
| Rabbit Polyclonal | Atlas | HPA029018 | IHC, WB |
| | Antibodies | | |
| Rabbit Polyclonal | Fitzgerald | 70R-1149 | WB |
| | Industries | | |
| | International | | |
| Rabbit Polyclonal | GenWay | 18-003-44307 | WB |
| | Biotech | | |
| Rabbit Polyclonal | GenWay | GWB-330500 | WB |
| | Biotech | | |
| Rabbit Polyclonal | Proteintech | 16675-1-AP | ELISA, WB, IP, |
| | Group | | IHC, IF |
| Rabbit Polyclonal | Bethyl | A302-889A-M | WB, IP |
| | Laboratories | | |
| Rabbit Polyclonal | Bethyl | A302-889A-T | WB, IP |
| | Laboratories | | |
| Rabbit Polyclonal | OriGene | TA590740 | WB, ELISA, IHC, |
| | Technologies | | IF |
| Rabbit Polyclonal | Bioss | bs-6511R | WB, IHC-P, IF |
| Mouse Monoclonal | Creative | MOB-1971z | WB, ELISA, IHC |
| | Biolabs | | |
| Rabbit Polyclonal | ProSci | 29-818 | ELISA, WB |
| Goat Polyclonal | Santa Cruz | sc-21564 | WB, IF, ELISA |
| Mouse Monoclonal | Santa Cruz | sc-166358 | WB, IP, IF, ELISA |

WB, Western blotting;
IHC, immunohistochemistry;
IF, immunofluorescence;
ICC, immunocytochemistry;
IP, immunoprecipitation;
ELISA, enzyme-linked immunosorbent assay.

Methods of generating antibodies (e.g., monoclonal or polyclonal antibodies) are well known in the art (e.g., Harlow and Lane, Antibodies: A Laboratory Manual, Cold Spring Harbor Laboratory, New York, 1988). For example, peptide fragments of AMFR can be conjugated to carrier molecules (or nucleic acids encoding such epitopes) can be injected into non-human mammals (e.g., mice or rabbits), followed by boost injections, to produce an antibody response. Serum isolated from immunized animals may be isolated for the polyclonal antibodies contained therein, or spleens from immunized animals may be used for the production of hybridomas and monoclonal antibodies. Antibodies can be further purified before use.

Aptamers used in the methods disclosed herein include single stranded nucleic acid molecule (e.g., DNA or RNA) that assumes a specific, sequence-specific shape and binds to the AMFR protein with high affinity and specificity. In another example, an aptamer is a peptide aptamer that binds to one of the AMFR protein with high affinity and specificity. Peptide aptamers include a peptide loop which is specific for the target protein attached at both ends to a protein scaffold. The scaffold may be any protein which is stable, soluble, small, and non-toxic. Peptide aptamer selection can be made using different systems, such as the yeast two-hybrid system or the Lex A interaction trap system.

In certain embodiments of the present invention, a kit may include a carrier means, such as a box, a bag, a vial, a tube, a satchel, plastic carton, wrapper, or other container. In some examples, kit components will be enclosed in a single packing unit, which may have compartments into which one or more components of the kit can be placed. In other examples, a kit includes one or more containers that can retain, for example, one or more biological samples to be tested. In some embodiments, a kit may include buffers and other reagents that can be used for the practice of a particular disclosed method. Such kits and appropriate contents are well known to those skilled in the art.

The present invention further provides methods for isolating iCSC or iLSCs or substantially homogeneous iCSC or iLSC populations from an established solid tumor or an established hematopoietic cancer. An embodiment of the methods includes a method for isolating AMFR-positive iCSC or iLSCs or substantially homogeneous iCSC or iLSC populations, which comprises the steps of: (a) preparing a sample of said solid tumor or said hematopoietic cancer; (b) contacting said sample with a binding agent that binds to AMFR on the cell surface; and (c) isolating tumor cells or cancer cells from said sample that express AMFR on the cell surface, thereby isolating said invasive tumor stem cell or said invasive leukemia stem cell.

In the above method, isolating AMFR-positive iCSCs or iLSCs or substantially homogeneous iCSC or iLSC populations involves the use of fluorescence-activated cell sorting (FACS), magnetic-assisted cell sorting, or any means that are capable of selecting cells based on specific protein epitopes present on the cell surface.

The technique of flow cytometry, such as fluorescence-assisted cell sorting (FACS) and the tumor-xenograft animal model are often used to enrich for specific CSC populations. This technique has the advantage of being able to simultaneously isolate phenotypically pure populations of viable normal and tumor cells for molecular analysis. Thus, flow cytometry allows us to test the functions of the cell populations and use them in biological assays in addition to studying their gene expression profiles. Furthermore, such cells can also be characterized in biological assays. For example, mesenchymal (stromal) cells can be analyzed for production of growth factors, matrix proteins and proteases, endothelial cells can be analyzed for production of specific factors involved in solid tumor growth support (such as neo-vascularization), and different subsets of tumor cells from a solid tumor can be isolated and analyzed for tumorigenicity, drug resistance and metastatic potential.

Diagnosis of Aggressive Solid Tumors or Hematopoietic Cancers

The present provides methods for diagnosing an aggressive solid tumor or an aggressive hematopoietic cancer comprising the steps of: (a) obtaining a first biological sample containing tumor or cancer cells from a first individual; (b) determining the frequency of said tumor or cancer cells with AMFR on their cell surface in said first biological sample; (c) comparing said frequency in said first biological sample with a second biological sample selected from the group consisting of an earlier obtained biological sample from said individual or a control biological sample obtained from a second individual without said solid tumor or hematopoietic cancer; and (d) determining if said frequency in said first biological sample is different (higher or lower) than that in said second biological sample.

The methods for diagnosing an aggressive solid tumor or an aggressive hematopoietic cancer can enable the prediction of clinical prognosis, including disease recurrence, metastasis, treatment response, and overall survival in any subject with a solid tumor or a hematopoietic cancer. Accordingly, the present invention can be used to screen subjects with solid tumor or hematopoietic cancer for poor clinical prognosis, including, for example, disease recurrence following treatments, which can direct treatment decisions and the choice of treatment modalities for subjects with said solid tumor or hematopoietic cancer. Thus, the subject and the caregiver can make better informed decisions of whether or not to perform surgery, neo-adjuvant (i.e., before surgery), adjuvant therapy (i.e., after surgery), including, without limitation, radiation treatment, chemotherapy treatment, treatment with biological agents, or hormone therapy, and/or other alternate treatment(s).

Disclosed methods involve determining the frequency of tumor or cancer cells with AMFR on their cell surface in an individual and then comparing the frequency to the frequency of said AMFR-positive tumor or cancer cells determined from an earlier obtained sample for the same individual or a control sample obtained from an individual without said solid tumor or hematopoietic cancer.

In a preferred embodiment, the frequency of AMFR-positive tumor or cancer cells in a large number of persons or tissues with said solid tumor or hematopoietic cancer and whose clinical prognosis data are available as measured using a tissue sample or biopsy or other biological sample such a cell, serum or blood can be used to determine a reference level. Thus, said frequency of AMFR-positive tumor or cancer cells in an individual determined by defining levels wherein said individuals whose tumors have said frequency of AMFR-positive tumor or cancer cells above that said reference level(s) are predicted as having a higher or lower risk of tumor aggressiveness, poor clinical prognosis or disease progression than those with expression levels below said reference level(s). Variation of levels of said frequency of AMFR-positive tumor or cancer cells from the reference range (either up or down) indicates that the individual has a higher or lower degree of aggressiveness or risk of poor clinical prognosis or disease progression than those with expression levels below said reference level(s).

In exemplary methods, determining the protein expression levels comprises the use of antibodies specific to said gene markers and immunohistochemistry staining on fixed (e.g., formalin-fixed) and/or wax-embedded (e.g., paraffin-embedded) prostate tumor tissues. Fixatives for tissue preparations or cells and antibody regents useful for this application have been described above.

Definitions

The term “stem cells” is well known in the art and denotes to cells that are capable of generating a plurality of progenies with varying proliferative and developmental potentials. Stem cells have extensive proliferative capacity and are capable of self-renewal (see, Potten et al., Development 110: 1001 (1990); U.S. Pat. Nos. 5,750,376, 5,851,832, 5,753,506, 5,589,376, 5,824,489, 5,654,183, 5,693,482, 5,672,499, and 5,849,553, all incorporated by reference). Cells in some animal tissues, such as bone marrow, gut, secretory glands, and skin, are constantly replenished from a small population of stem cells in each tissue. Thus, the maintenance of tissues (whether during normal life or in response to injury and disease) depends upon the replenishing of the tissues from precursor cells in response to specific developmental signals.

As used herein the terms “cancer stem cells (CSCs)” or “tumor stem cells” are used interchangeably and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of CSCs confer them the ability to form palpable tumors upon serial transplantation into an immunocompromised mouse compared to the majority of tumor cells that fail to form tumors. CSCs undergo self-renewal versus differentiation in a chaotic manner to form tumors with abnormal cell types that can change over time as mutations occur.

As used herein “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor.

As used herein, the terms “stem cell cancer marker(s),” “cancer stem cell marker(s),” “tumor stem cell marker(s),” or “solid tumor stem cell marker(s)” refer to a gene or genes or a protein, polypeptide, or peptide expressed by the gene or genes whose expression level, alone or in combination with other genes, is correlated with the presence of tumorigenic cancer cells compared to non-tumorigenic cells. The correlation can relate to either an increased or decreased expression of the gene (e.g. increased or decreased levels of mRNA or the peptide encoded by the gene).

As used herein, the term “Enriched”, as in an enriched population of cells, can be defined based upon the increased number of cells having a particular marker in a fractionated set of cells as compared with the number of cells having the marker in the unfractionated set of cells. However, the term “enriched can be preferably defined by tumorigenic function as the minimum number of cells that form tumors at limit dilution frequency in test mice.

As used herein, the term “podosomes” or “invadopodia” refer to transient actin-based protrusions in motile cells or invasive cancer cells that mediate focal degradation of ECM by the localized proteolytic activity of proteases.

As used herein, the term “epithelial-mesenchymal transition (EMT))” refers to the ability of epithelial cells to transition into mesenchymal cells by obtaining their characteristics. EMT does not occur in normal cells except during the process of embryogenesis. Epithelial cells, which are bound together tightly and exhibit polarity, change into mesenchymal cells that are bound together more loosely, exhibit a loss of polarity, and have the ability to move. These mesenchymal cells can spread into tissues around the primary tumor, and also separate from the tumor, invade blood and lymph vessels, and move to new locations where they divide and form additional tumors. Drug resistance, metastasis, or recurrence of cancer can be explained by such additional tumor formation.

As used herein, the present invention the present invention provides substantially homogeneous iCSC or iLSC populations comprising said iCSCs or iLSC of the present invention. “Substantially homogeneous” means that, when immunodeficient animals are grafted with 1000 cells, 100 cells, or 10 cells and analyzed for the frequency of formation of cancer cell populations using Extreme Limiting Dilution Analysis (Hu Y & Smyth G K., J Immunol Methods. 2009 Aug. 15; 347(1-2): 70-8) utilizing, for example, the method described in Hu Y & Smyth G K., J Immunol Methods. 2009 Aug. 15; 347 (1-2):70-8 or Ishizawa K & Rasheed Z A. et al., Cell Stem Cell. 2010 Sep. 3; 7(3):279-82, the frequency of cancer stem cells is 1/20 or more, preferably 1/10 or more, more preferably 1/5 or more, even more preferably 1/3 or more, still more preferably 1/2 or more, and yet more preferably 1/1.

As used herein, the term “expansion of iCSC or iLSC” refers to, for example, proliferation by spheroid culture or grafting and passaging in non-human animals, but is not particularly limited thereto.

As used herein, the term “aggressive solid tumors” refers to those solid tumors associated with high likelihoods of invading into surrounding tissues and/or developing metastatic lesions at distant sites.

As used herein, the term “aggressive hematopoietic cancers” refers to those hematopoietic cancers associated with high likelihoods of causing severe damage to the bone marrow and/or invading the liver, the lymph nodes, the central nervous system or any tissues outside the bone marrow.

As used herein, the term “clinical prognosis” refers to the outcome of subjects with solid tumors or blood cancers comprising the likelihood of tumor recurrence, survival, disease progression, and response to treatments. The recurrence of tumor or cancer after treatment is indicative of a more aggressive cancer, a shorter survival of the host (e.g., cancer patients), an increased likelihood of an increase in the size, volume or number of tumors, and/or an increased likelihood of failure of treatments.

As used herein, the term “predicting clinical prognosis” refers to providing a prediction of the probable course or outcome of pancreatic cancer, including prediction of metastasis, multidrug resistance, disease free survival, overall survival, recurrence, etc. The methods can also be used to devise a suitable therapy for cancer treatment, e.g., by indicating whether or not the cancer is still at an early stage or if the cancer had advanced to a stage where aggressive therapy would be ineffective.

“AMFR” refers to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 60% nucleotide sequence identity, 65%, 70%, 75%, 80%, 85%, 90%, preferably 91%, 92%, 93%, 94%, 95%, 96%, 97%, 98% or 99% or higher nucleotide sequence identity, preferably over a region of at least about 10, 15, 20, 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. Truncated and alternatively spliced forms of these antigens are included in the definition.

As used herein, the term “metastasis” refers to a process where cancer spreads or travels from the primary site to another location in the body, resulting in development of similar cancer lesions at the new site. “Metastatic” or “metastasizing” cell refers to a cell that has left the primary site of the disease due to loss of adhesive contact to adjacent cells and has invaded into neighboring body structures via blood or lymphatic circulation.

As used herein, the term “recurrence” refers to that, after partial resection of an organ to remove a malignant tumor from a cancer patient, or after postoperative chemotherapy, the same malignant tumor has reappeared in the remaining organ.

The details of one or more embodiments of the invention have been set forth in the accompanying description above. Although any methods and materials similar or equivalent to those described herein can be used in the practice or testing of the present invention, the preferred methods and materials are now described. Other features, objects, and advantages of the invention will be apparent from the description and from the claims.

In the specification and the appended claims, the singular forms include plural referents. Unless defined otherwise in this specification, all technical and scientific terms used herein have the same meaning as commonly understood by one of ordinary skill in the art to which this invention belongs. All patents and publications cited in this specification are incorporated by reference.

### EXAMPLES

The following examples are given for illustrative purposes only and are not intended to be limiting unless otherwise specified. It should be appreciated by those of skill in the art that the techniques disclosed in the examples which follow represent techniques discovered by the inventors to function well in the practice of invention, and thus can be considered to constitute preferred modes for its practice. Those of skill in the art should appreciate that many changes can be made in the specific embodiments which are disclosed and still obtain a like or similar result without departing from the spirit and scope of the invention.

### Example 1

This example describes that cell-surface AMFR marks a subpopulation of CSCs in gastric cancer and pancreatic cancer.

We analyzed the expression of cell surface AMFR in gastric cancer and pancreatic cancer cells by the fluorescence-assisted cell sorting (FACS) analysis. We measured the proportion of primary gastric cancer-derived AGS cells (American Type Culture Collections) and metastatic gastric cancer SNU-16 cells (American Type Culture Collections) that expressed the surface marker CD90, which have been shown to contain the enriched CSCs in gastric cancer (Jiang et al., 2012). We also measured the proportion of primary pancreatic cancer-derived PANC-1 cells and metastatic pancreatic cancer-derived AsPC-1 cells (both from American Type Culture Collections) that expressed that surface markers CD133 and CD90, which have been shown to contain the enriched CSCs in pancreatic cancer (Wang et al., 2013). For the FACS analysis, cells were dissociated, antibody-labeled (1-2 μg per 10⁶ cells×1 hour) and resuspended in HBSS/2% FBS as previously described (Hermann et al., 2007; Li et al., 2007). The antibodies used included anti-CD90 antibody PE-anti-CD90 (BD Biosciences) and anti-AMFR (PA5-12051; Thermo Scientific) in conjunction with Alexa Fluor 488-anti-mouse IgG (Invitrogen). Flow cytometry was done using a FACSAria III (BD Biosciences).

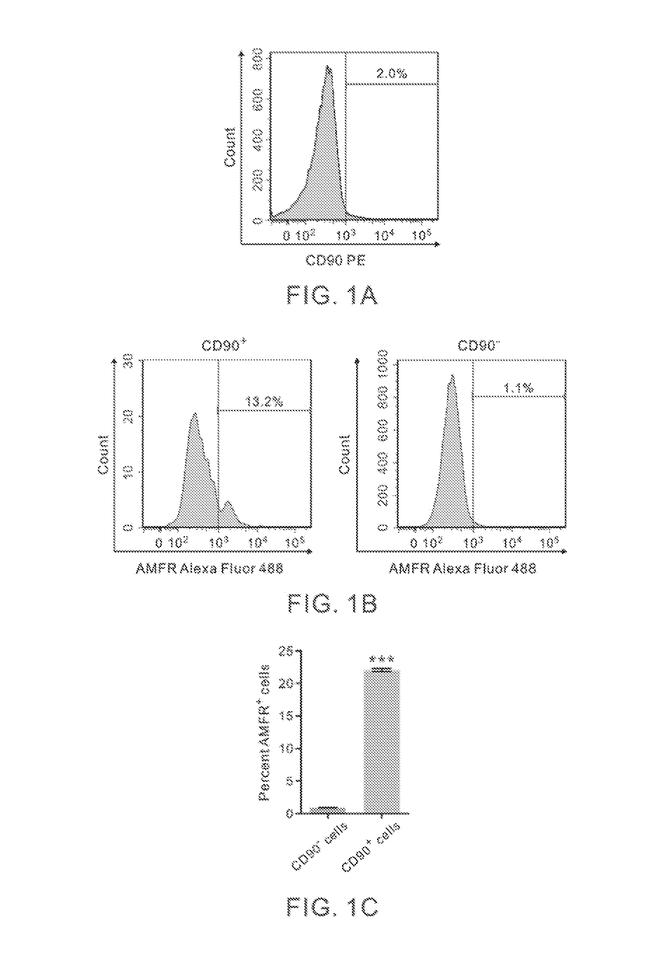

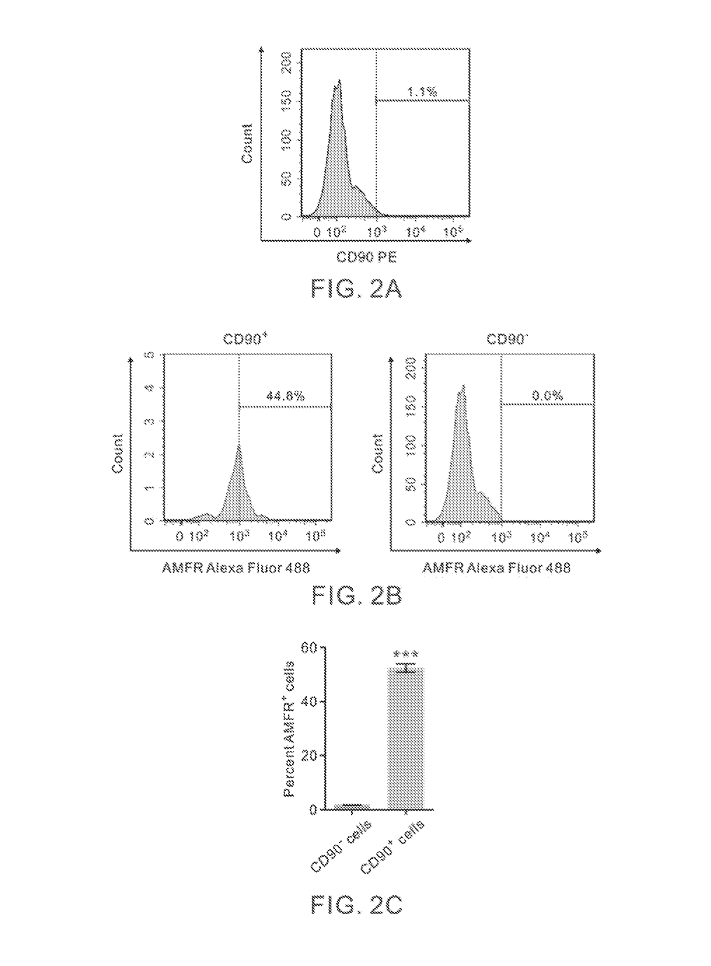

As shown in FIG. 1A-1C, a considerable proportion (22.07% on average) of CD90⁺ AGS cells were positive for the cell surface AMFR while very few (0.97% on average) of cells in the other subpopulations expressing AMFR on their cell surface. Likewise, as shown in FIG. 2A-2C, in metastatic gastric cancer SNU-16 cells, a large proportion (52.47% on average) of the CD90⁺ cells were positive for cell-surface AMFR, whereas cell-surface AMFR was detectable in only 1.9% of CD90⁻ cells. These results together indicate that gastric cancer CSCs exclusively express cell-surface AMFR than their non-CSC counterparts, supporting it as a CSC-specific marker in both primary and metastatic gastric cancer.

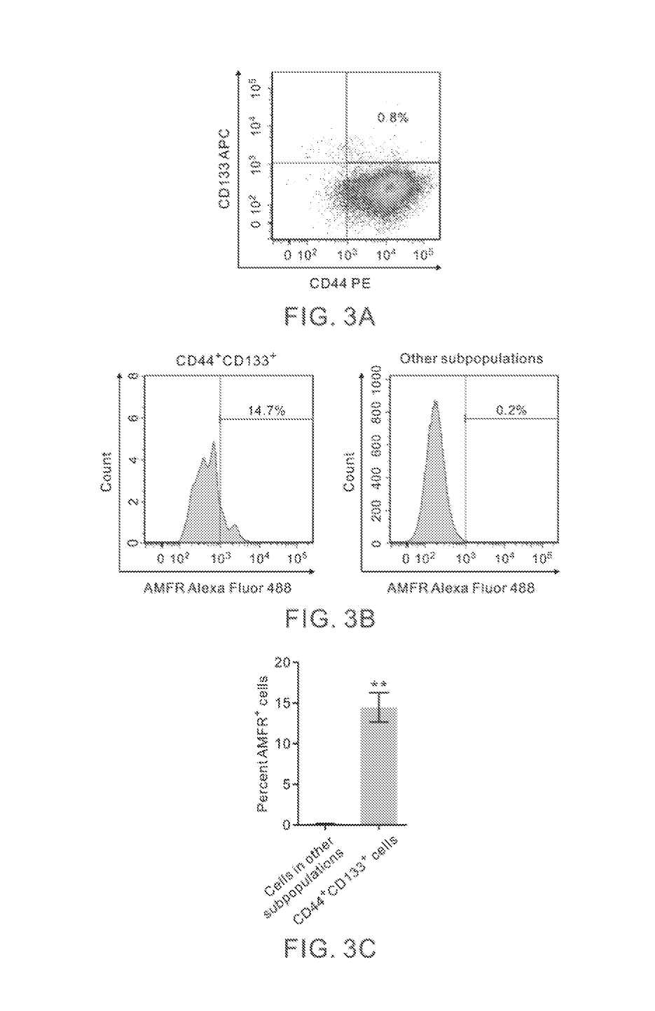

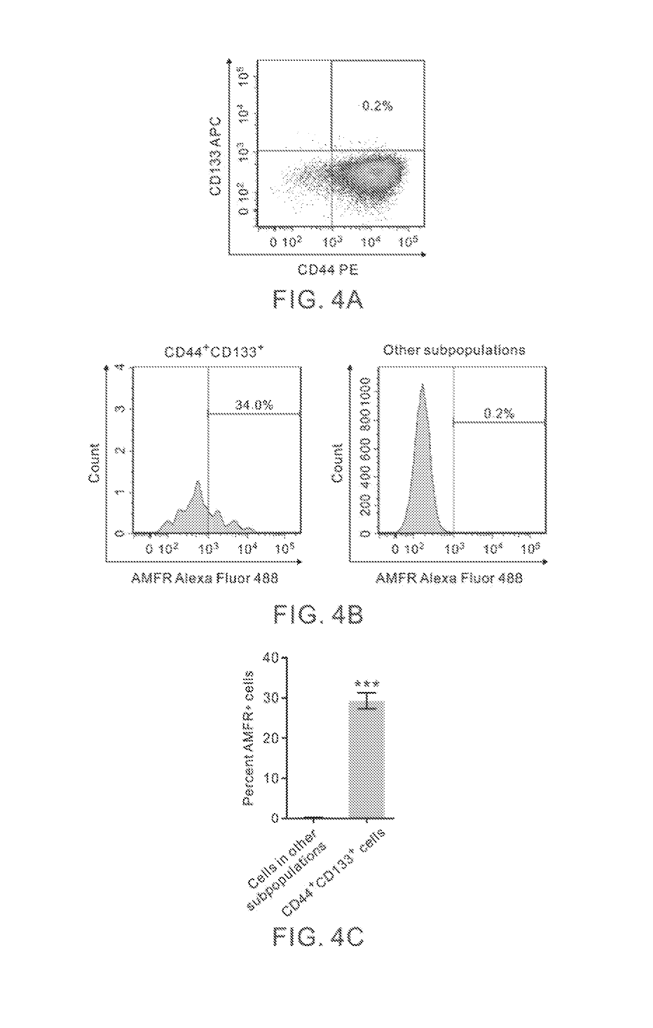

As shown in FIG. 3A-3C, a significant proportion (14.47% on average) of CD44⁺CD133⁺ pancreatic cancer PANC-1 cells were positive for the cell surface AMFR while very few (0.17% on average) of cells in the other subpopulations were AMFR-positive. Similarly, as shown in FIG. 4A-4C, in metastatic pancreatic cancer AsPC-1 cells, a considerable proportion (29.27% on average) of the CD44⁺CD133⁺ cells were positive for AMFR, whereas only 0.17% of the cells in the other subpopulations were AMFR-positive. These results indicate that pancreatic CSCs exclusively express AMFR on their surface than their non-stem cell counterparts, lending support to it as a CSC-specific marker in both primary and metastatic pancreatic cancers.

### Example 2

This example describes that cell-surface AMFR marks a subpopulation of glioma stem cells.

We analyzed the expression of cell surface AMFR in malignant glioma by the FACS analysis. We measured the proportion of two malignant glioma (glioblastoma) cell lines, U-87MG and Hs-683 cells (both obtained from American Type Culture Collections) that expressed the surface marker CD133, which have been shown to contain the enriched glioma stem cells (Singh et al., 2004). For the FACS analysis, cells were dissociated, antibody-labeled (1-2 μg per 10⁶ cells×1 hour) and resuspended in HBSS/2% FBS as previously described (Hermann et al., 2007; Li et al., 2007). The antibodies used included APC-anti-CD133 (Miltenyi Biotec, Bergisch Gladbach, Germany) and anti-AMFR (PAS-12051; Thermo Scientific) in conjunction with Alexa Fluor 488-anti-mouse IgG (Invitrogen). Flow cytometry was done using a FACSAria III (BD Biosciences).

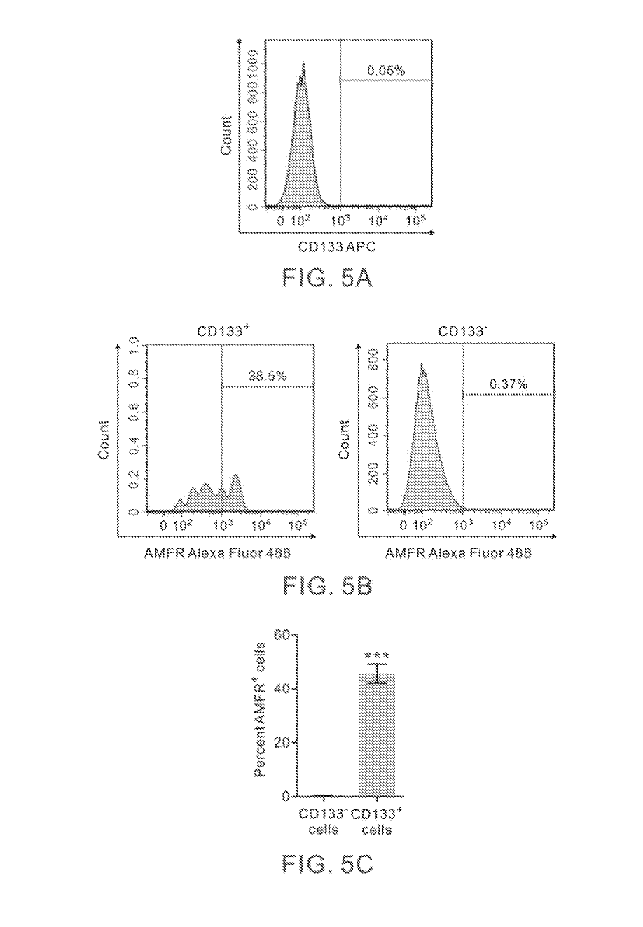

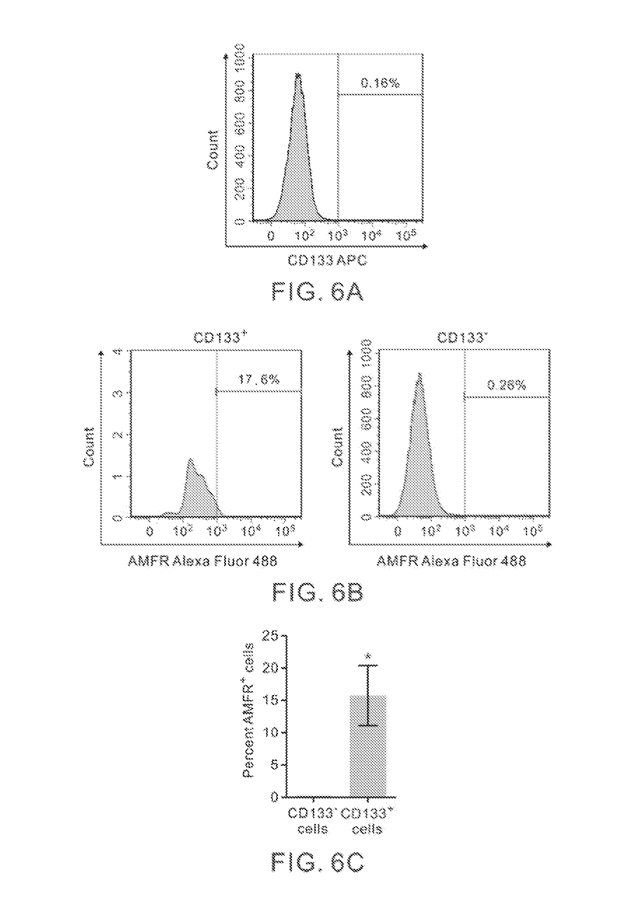

As shown in FIG. 5A-5C, a large proportion (45.73% on average) of CD133⁺ U-87MG cells were positive for cell surface AMFR while very few (0.47% on average) of CD133⁻ cells expressed AMFR on their cell surface. Similarly, as shown in FIG. 6A-6C, in another malignant glioma cell line Hs-683 cells, many (15.76% on average) of the CD133⁺ cells were positive for AMFR, whereas only an average of 0.28% of the CD133⁻ cells in the other subpopulations were AMFR-positive. These results indicate that malignant glioma stem cells exclusively express AMFR on their cell surface, supporting its role as a glioma stem cell-specific marker.

### Example 3

This example describes that cell-surface AMFR marks a subpopulation of CSCs in lung cancer and prostate cancer.

We analyzed the expression of cell surface AMFR in lung cancer and prostate cancer cells by the FACS analysis. We measured the proportion of primary lung adenocarcinoma-derived A-549 cells or a primary lung squamous cell carcinoma-derived line, NCI-H520 cells (both obtained from American Type Culture Collections) that expressed CD133, which have been shown to contain the enriched CSCs in non-small cell lung cancer (NSCLC) (Eramo et al., 2008). We also measured the proportion of primary prostate cancer-derived 22Rv-1 cells or metastatic prostate cancer PC-3 cells (both obtained from American Type Culture Collection) that expressed that surface markers CD44 and CD133, which have been shown to contain the enriched CSCs in prostate cancer (Dubrovska et al., 2009; Richardson et al., 2004). For the FACS analysis, cells were dissociated, antibody-labeled (1-2 μg per 10⁶ cells×1 hour) and resuspended in HBSS/2% FBS as previously described (Hermann et al., 2007; Li et al., 2007). The antibodies used included APC-anti-CD133 (Miltenyi Biotec, Bergisch Gladbach, Germany), PE-anti-CD44 (BD Biosciences), and anti-AMFR (PAS-12051; Thermo Scientific) in conjunction with Alexa Fluor 488-anti-mouse IgG (Invitrogen). Flow cytometry was done using a FACSAria III (BD Biosciences).

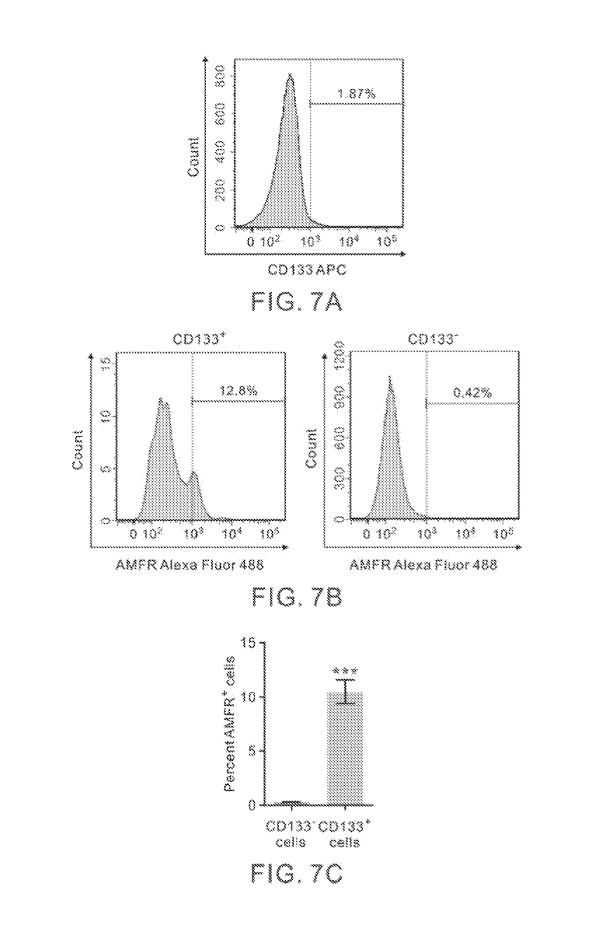

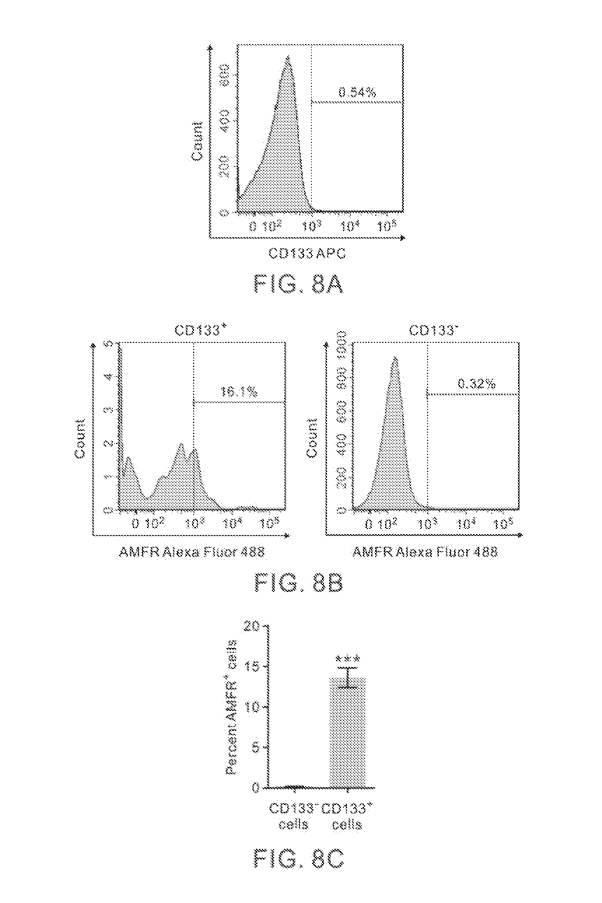

As shown in FIG. 7A-7C, a significant proportion (10.47% on average) of CD133⁺ A-549 cells were positive for cell surface AMFR while very few (0.3% on average) of CD133⁻ cells expressed AMFR on their cell surface. Similarly, as shown in FIG. 8A-8C, in another lung cancer cell line NCI-H520 cells, which was derived from a primary squamous cell carcinoma of the lung, many (13.63% on average) of the CD133⁺ cells were positive for cell-surface AMFR, whereas only 0.2% of CD133⁻ cells expressed cell-surface AMFR. These results indicate that CSCs in both adenocarcinoma and squamous cell carcinoma of the lung exclusively express AMFR on their cell surface, supporting its role as a CSC-specific marker in NSCLC.

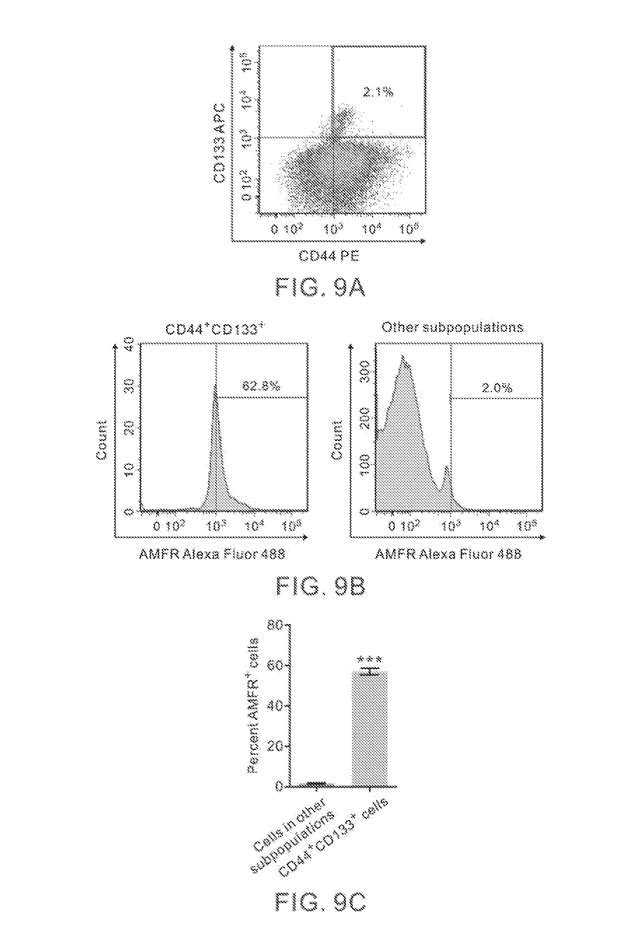

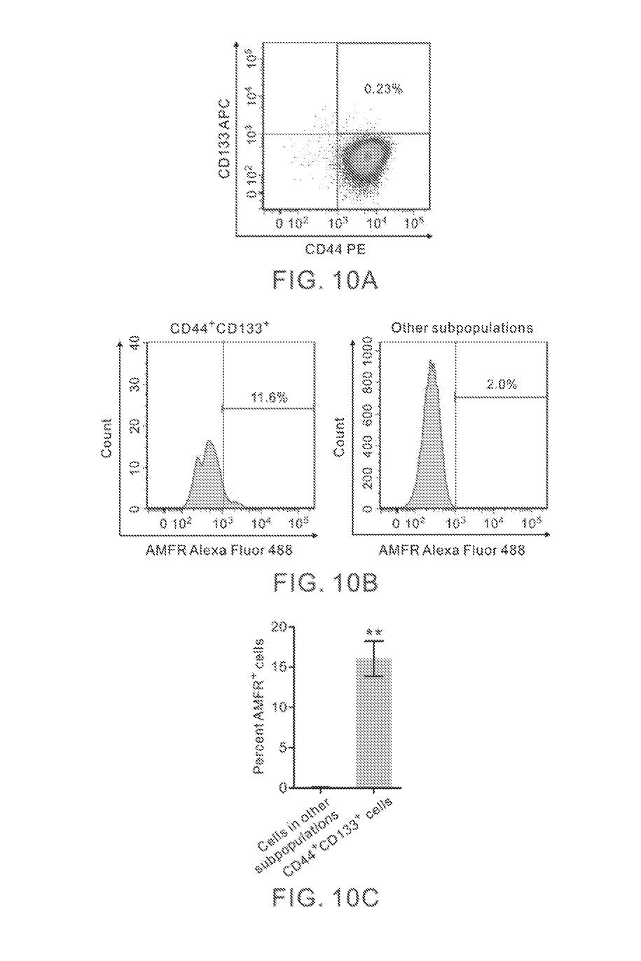

As shown in FIG. 9A-9C, the majority (57.0% on average) of CD133⁺CD44⁺ 22Rv-1 cells were positive for AMFR while only 1.63% of cells in the other subpopulations expressed AMFR on their cell surface. Similarly, as shown in FIG. 10A-10C, in metastatic prostate cancer-derived line PC-3 cells, a considerable proportion (16.03% on average) of the CD133⁺CD44⁺ cells were positive for AMFR, whereas very few (0.1%) of the other subpopulations of cells expressed AMRF. These results indicate that CSCs in primary or metastatic prostate cancer exclusively express AMFR on their cell surface, supporting its role as a prostate CSC-specific marker.

### Example 4

This example describes that cell-surface AMFR marks a significant proportion of LSCs in acute myeloid leukemia.

We analyzed the expression of cell surface AMFR in AML cells by the FACS analysis. We measured the proportion of AML THP-1 cells and HL-60 cells (both obtained from American Type Culture Collections) that expressed CD34 but lacked the expression of CD38, which have been shown to contain the enriched LSCs in AML (van Rhenen et al., 2005). For the FACS analysis, cells were dissociated, antibody-labeled (1-2 μg per 10⁶ cells×1 hour) and resuspended in HBSS/2% FBS as previously described (Hermann et al., 2007; Li et al., 2007). The antibodies used included FITC-CD34, APC-CD38 (both from BD Biosciences) and anti-AMFR (PAS-12051; Thermo Scientific) in conjunction with Alexa Fluor 488-anti-mouse IgG (Invitrogen). Flow cytometry was done using a FACSAria III (BD Biosciences).

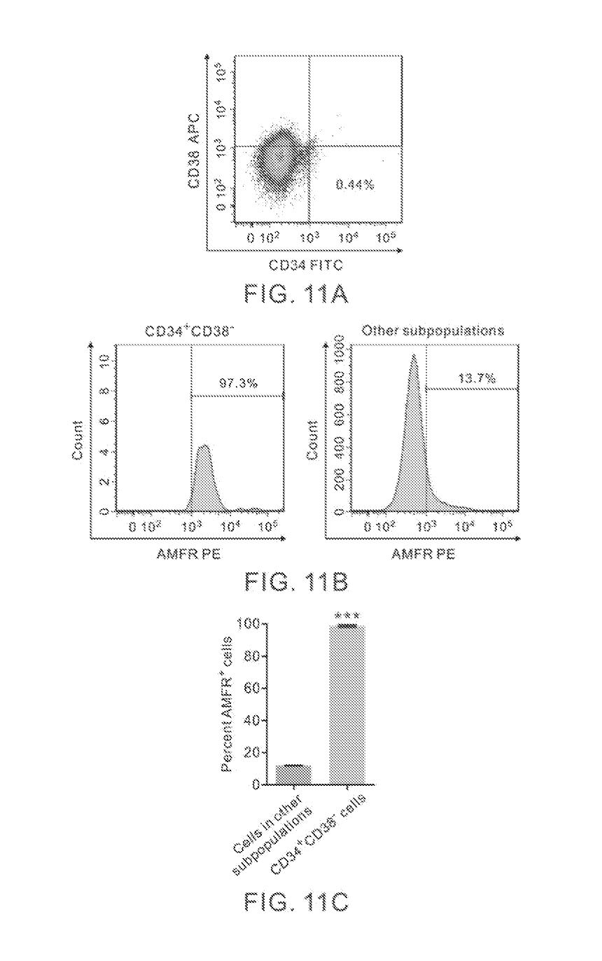

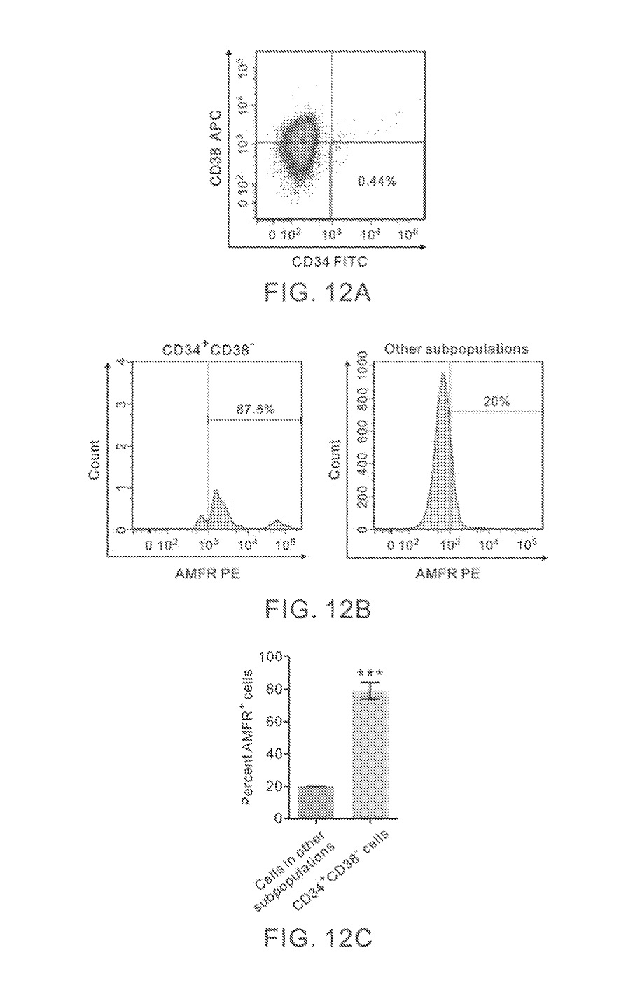

As shown in FIG. 11A-11C, the vast majority (98.67% on average) of CD34+CD38− THP-1 cells were positive for the cell surface AMFR while only a few (12.17% on average) of the cells in the other subpopulations expressing AMFR on their cell surface. Similarly, as shown in FIG. 12A-12C, in another AML line HL-60 cells, the majority (78.97% on average) of the CD34+CD38− cells were positive for cell-surface AMFR, whereas only a few 19.93% of the cells in the other subpopulations expressed cell-surface AMFR. These results indicate that leukemia stem cells exclusively express AMFR on their cell surface. These results suggest that cell-surface AMFR is not only a CSC-specific marker in solid tumors but also a LSC-specific marker in hematologic malignancies like AML.

### Example 5

This example describes AMFR as a specific marker of mesenchymal-like and invasive CSCs.

We isolated three subpopulations of cells from pancreatic cancer PANC-1 cells according to the expression of the cell-surface markers CD133 and CD44, which have been shown to contain the enriched CSCs in pancreatic cancer (Wang et al., 2013), and AMFR, including CD133⁺CD44⁺AMFR⁺ cells (representing AMFR-positive CSC s), CD133⁺CD44⁺AMFR⁻ cells (representing AMFR-negative CSCs), and cells in the other subpopulations (representing non-CSC cancer cells). In brief, PANC-1 cells (American Type Culture Collections) were dissociated, antibody-labeled (1-2 μg per 10⁶ cells×1 hour) and resuspended in HBSS/2% FBS as previously described (Al-Hajj et al., 2003; Li et al., 2007). The antibodies used included PE-anti-CD44, APC-anti-CD133 (Multenyi Biotec) and anti-AMFR (LifeSpan BioSciences) in conjunction with Alexa Fluor 488-anti-mouse IgG (Invitrogen). Cell sorting was performed using FACSAria™ III cell sorter (BD Biosciences). Of note, the AMFR antibody used recognizes an epitope located between amino acids 4-33 located within the transmembrane domain (amino acids 1-308) of human AMFR, whereby it does not interfere with its ability to bind to AMF, which is mediated through the C-terminal part (amino acids 309-643) of AMFR (Haga et al., 2006).

Cells in the respective cell subpopulation were collected and analyzed for their transcript levels of a panel of gene markers widely associated with EMT or pleuripotency by quantitative real-time PCR (qRT-PCR), which was performed on the amplified RNA using the LightCycler FastStart DNA MASTERPLUS SYBR Green I Kit (Roche Diagnostics GmbH) and Q-Cycler 96 (Hain Lifescience). Oligonucleotide primers were designed using Primer Bank (http://pga.mgh.harvard.edu/primerbank/index.html). Cells in the respective cell subpopulations collected were also analyzed for their invasive capacities using the modified Boyden chamber assay. Briefly, the freshly sorted cells were seeded on upper wells of 48-well Neuro Probe AP48 chemotaxis chambers (Neuro Probe). The 8-μm pore polycarbonate filter was coated with a thin layer of type I collagen (BD Biosciences) with pancreatic stellate cells (PSCs)-conditioned media in the lower wells as chemoattractant. After an incubation period of 12 hours, the uninvaded cells were wiped off the top side of the filter and the invaded cells were fixed, stained with CYTOX-green (Invitrogen) and counted using a fluorescence microscope.

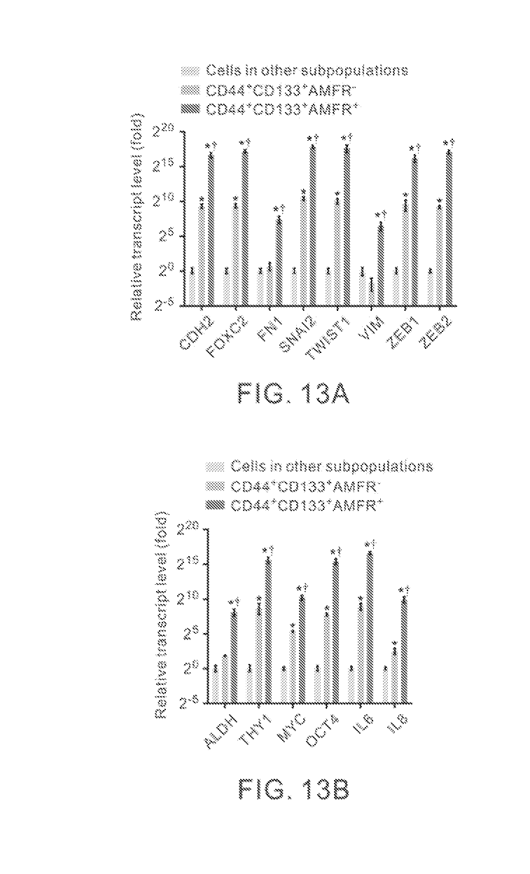

As shown in FIG. 13A, the relative transcript levels of a panel of EMT marker genes, including CDH2 (encoding N-cadherin), FOXC2 (encoding forkhead box C2), FN1 (encoding fibronectin), SNAI2 (encoding Slug), TWIST1 (encoding Twist), VIM (encoding vimentin), ZEB1 (encoding Zinc finger E-box binding homeobox-1), and ZEB2 (encoding Zinc finger E-box binding homeobox-2), are significantly up-regulated in CD133⁺CD44⁺AMFR⁺ PANC-1 cells compared with CD133⁺CD44⁺AMFR⁻ cells and cells in the other subpopulations.

As shown in FIG. 13B, the relative transcript levels of a panel of genes that are frequently associated with embryonic stem cells or pleuripotency, including ALDH (encoding aldehyde dehydrogenase), THY1 (encoding CD90), MYC (encoding c-Myc), OCT4 (encoding Oct-4), IL6 (encoding interleukin 6), and IL8 (encoding interleukin 8), are significantly up-regulated in CD133⁺CD44⁺AMFR⁺ PANC-1 cells compared with CD133⁺CD44⁺AMFR⁻ cells and cells in the other subpopulations.

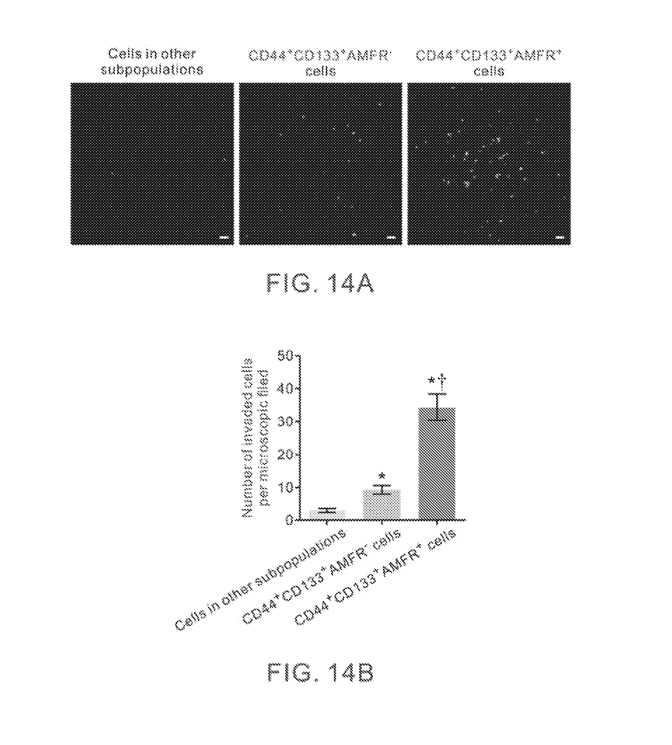

To functionally validate that AMFR-positive cells are mesenchymal-like and pro-invasive, we compared the invasive capacity of AMFR-positive PANC-1 cells with that in AMFR-negative cells. As shown in FIG. 14A-14B, CD133⁺CD44⁺AMFR⁺ PANC-1 cells (representing AMFR-positive CSCs) indeed had a remarkably higher capacity to invade through collagen than that of AMFR⁻ CSCs. By contrast, the cells in the other cell subpopulations (representing NSCCs) could barely invade the collagen matrix. Taken together, the above gene expression and functional data suggest that AMFR marks a population of CSCs that have mesenchymal-like properties and are highly invasive.

### Example 6

This example describes that AMFR is present on the surface of the invadopodia in mesenchymal-like CSCs.

Invadopodia are important cellular structure that mediate cancer cell invasion. Specifically, invadopodia are transient actin-based protrusions in invasive cancer cells that mediate focal degradation of extracellular matrix (ECM) by the localized proteolytic activity of proteases (Chen, 1989; Paz et al., 2014). Cancer cells use invadopodia during mesenchymal-type migration to degrade and invade extracellular matrix structures. Interestingly, AMFR has recently been found to stably localize to the lipid raft caveolae and partially colocalize with its constituent protein caveolin-1 (Benlimame et al., 1998). Consistently, caveolin-1 has been reported to serve as a negative regulator of the lipid-raft-dependent uptake of AMFR (Le et al., 2002). Some recent research also suggests that lipid rafts are required for the assembly and function of invadopodia in cancer cells. Consistently, caveolin-1 accumulates at invadopodia and its down-regulation inhibits Invadopodia-mediated ECM degradation (Yamaguchi et al., 2009). Depletion of caveolin-1 disrupts the association of essential components of invadopodia, including Src kinases, β1-integrin and urokinase receptor (uPAR), thereby compromising the migration of cancer cells on ECM (Wei et al., 1999). Taken together these findings, we consider the possibility that AMFR may specifically exist in the caveolae on the membrane of invadopodia in mesenchymal-like CSCs and contribute to their formation and functions, thereby promoting the invasive behaviors of mesenchymal-like CSCs and promoting cancer metastasis. To address this possibility, we isolated three subpopulations of cells from pancreatic cancer PANC-1 cells according to the expression of CD44, CD133 and AMFR, including CD44⁺CD133⁺AMFR⁺ cells (representing AMFR-positive CSCs), CD44⁺CD133⁺AMFR⁻ cells (representing AMFR-negative CSCs), and cells in the other subpopulations (representing non-CSC cancer cells). To induce invadopodium formation, we plated each of the three subpopulations of cells onto high-density fibrillar collagen (HDFC), which consists of a thin layer of densely packed fibrillar collagen type I compressed by centrifugation (Artym et al., 2015). HDFC can more potentially induce cellular invadopodia formation in a variety of cancer cells compared with gelatin which is used in the standard invadopodia assay. We immuno-stained the cells seeded on HDFC with antibodies against the invadopodium markers F-actin (Alexa Fluor-647 Phalloidin; Invitrogen) and cortactin (Abcam) as well as AMFR (Thermo Scientific) and evaluated the staining patterns using confocal imaging analysis (LSM 5 Pascal Confocal microscopy, Zeiss).

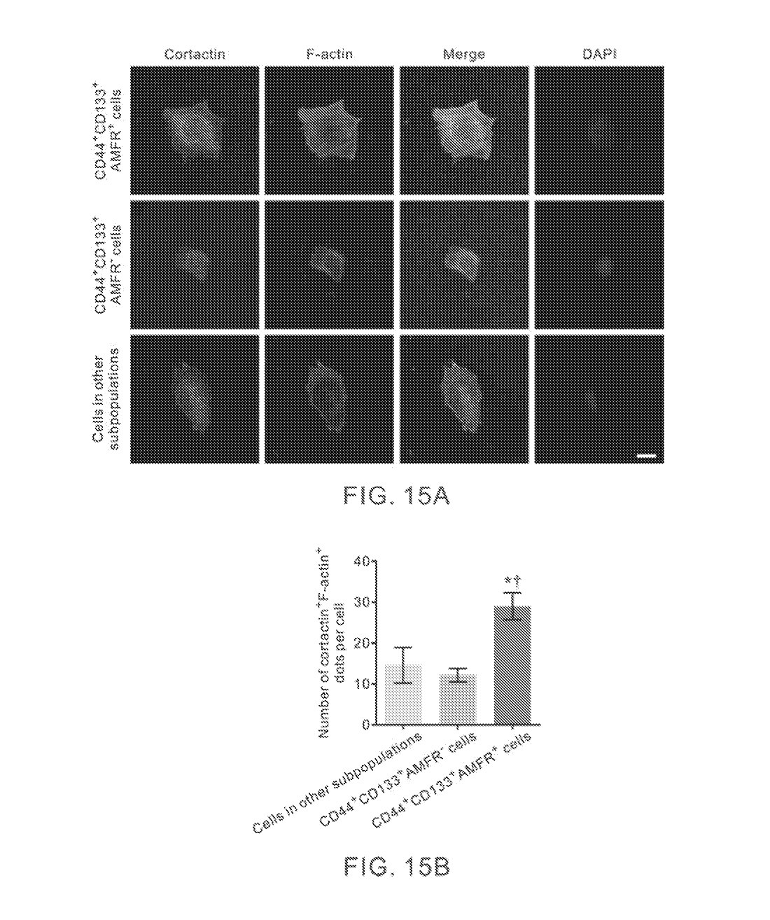

As shown in FIG. 15A-15B, CD44⁺CD133⁺AMFR⁺ PANC-1 cells exhibited more invadopodia, identified as actin-cortactin-rich aggregates associated with cell membrane adherent to HDFC, than CD44⁺CD133⁺AMFR⁻ cells or cells in the other subpopulations, suggesting that AMFR-positive CSCs have a potent ability to induce invadopodia formation in response to extracellular matrices like HDFC, whereas AMFR-negative CSCs or NSCCs are much less able to do so.

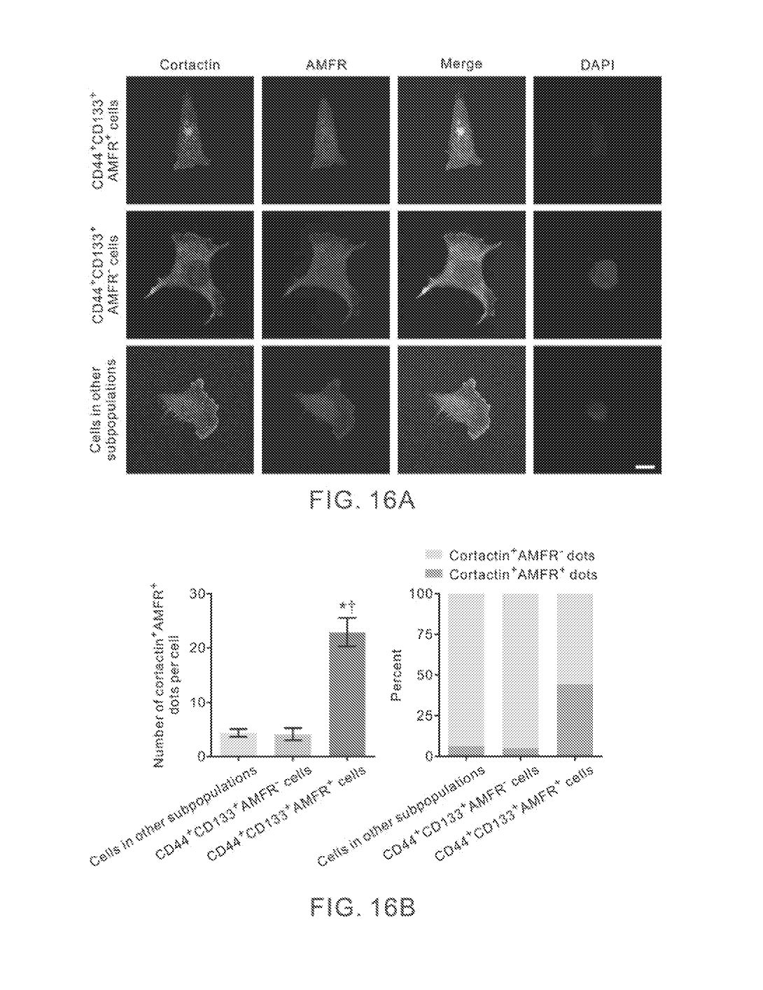

As shown in FIG. 16A-16B, in CD44⁺CD133⁺AMFR⁺ PANC-1 cells, AMFR was found colocalize with the invadopodia marker cortactin in the dot-like invadopodia structures that protruded from the undersurface of the CSCs into the underlying HDFC. These findings lend support to the hypothesis that AMFR is uniquely present in the invadopodia of CSCs.

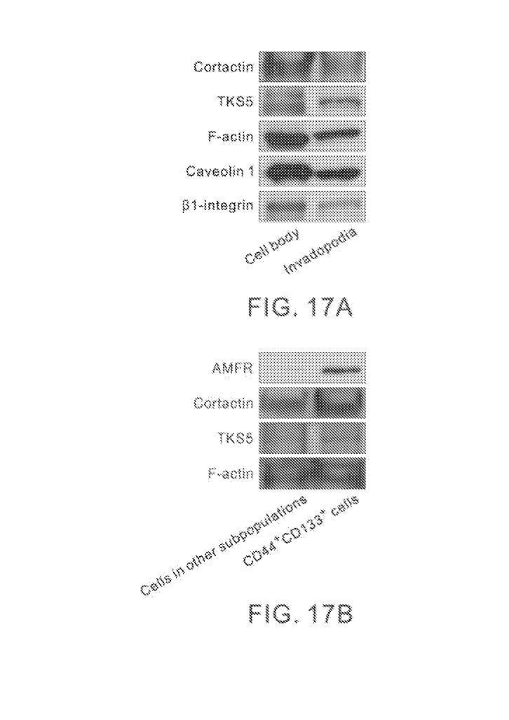

To further verify that AMFR exists specifically on the invadopodia of CSCs, we isolated the invadopodium proteins using a previously reported cell fractionation protocol in which cells adherent to HDFC were sheared away from the surface of the HDFC matrix, permitting the invadopodia that remained embedded in the HDFC stratum to be subsequently extracted to obtain an enriched invadopodia fraction (Attanasio et al., 2011; Bowden et al., 1999). Using this approach, we were able to fractionate PANC-1 cells that were seeded on HDFC into the invadopodia fraction and the cell body fraction. As shown in FIG. 17A, using immunoblotting analysis on the proteins known to be enriched in the invadopodia, including cortactin, TKS-5, β-actin, caveolin-1 (a marker of lipid rafts) and β1-integrin, we verified that we could isolate the enriched proteins from each fractions. We then compared the protein abundance of AMFR in the invadopodia of CD44⁺CD133⁺ PANC-1 cells and the cells in the other cell subpopulations (i.e., non-CSCs or NSCCs). As shown in FIG. 17B, AMFR indeed was indeed predominantly present in the invadopodia of CD44⁺CD133⁺ PANC-1 CSCs, whereas it is scarce in the invadopodia of NSCCs.

### Example 7

The example describes that AMFR is present in the invadopodia or podosomes of LSCs.

In Example 4, we demonstrated that AMFR is exclusively expressed by CD34⁺CD38⁻ LSCs in AML cells, including THP-1 cells (representing acute monocytic leukemia) and HL-60 cells (representing acute promyeoblastic leukemia). Published studies revealed that podosomes, which are the functional equivalents of invadopodia in normal motile cells, mediate the invasive behaviors of leukocytes such as macrophages, lymphocytes and dendritic cells (Carman et al., 2007; Linder, 2009; Linder et al., 2000; Olivier et al., 2006). Specifically, podosomes mediate the migration and matrix degradation abilities of macrophages (Cougoule et al., 2010) and the ability of lymphocytes to penetrate the endothelium though transcellular diapedesis (Carman et al., 2007). Our findings in Example 6 that AMFR is mainly present in the invadopodia of mesenchymal-like CSCs, together with the similarity between podosomes and invadopodia, raised the possibility that AMFR may also exist on the surface of the podosomes in LSCs and mediate their formation and functions. To address this possibility, we isolated CD34⁺CD38⁻ LSCs from THP-1 cells and cells in the other populations (representing non-stem like AML cells). To induce invadopodium formation in the isolated cells, we treated them with phorbal 12-myristate 13-acetate (PMA; 5 nM×72 hours) and then seeded them onto high-density fibrillar collagen (HDFC) using the methods described in Example 6. We then immunostained the cells seeded on HDFC with antibodies against the posodome markers F-actin (Alexa Fluor-647 Phalloidin; Invitrogen) and cortactin (Abcam) as well as AMFR (Thermo Scientific) and evaluated the staining patterns using confocal imaging analysis.

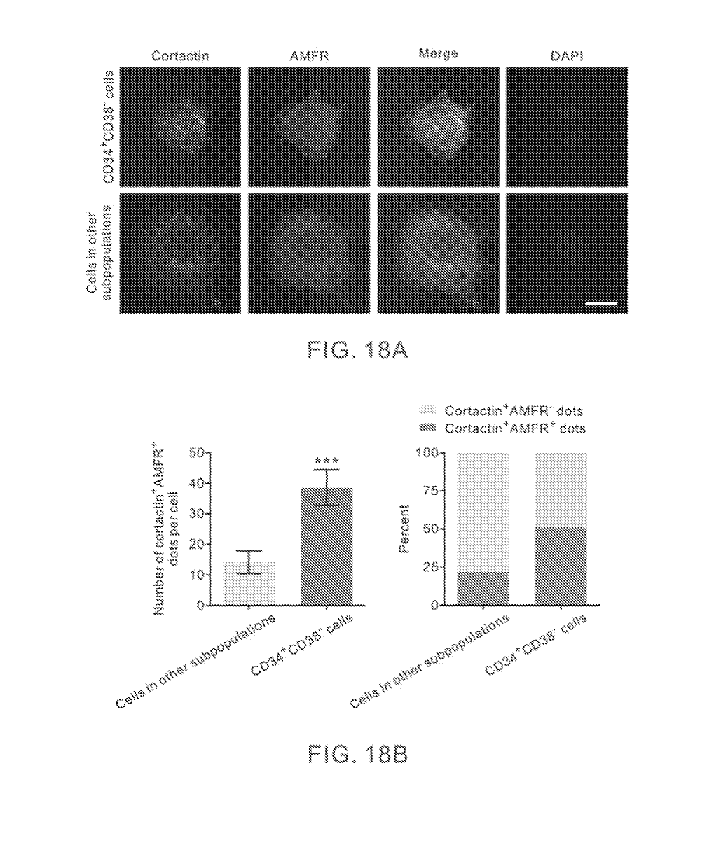

As shown in FIG. 18A-18B, in CD34⁺CD38⁻ THP-1 cells, AMFR was found colocalize with the invadopodia and podosome marker cortactin in the dot-like structures that protruded from the undersurface of the LSCs into the underlying HDFC. In comparison, there are much less numbers of AMFR/cortactin-colocalized structures in the cells in the other subpopulations (representing non-LSCs). These findings support that AMFR is uniquely present on the invadopodia or podosomes of LSCs.

### Example 8

The example describes that AMFR contribute to CSC-mediated cancer progression and metastasis.

In our findings in Example 7, we have provided compelling evidences showing that AMFR is present in the invadopodium of CSCs. A growing body of evidence revealing that invadopodia exist in vivo and may play a critical role for tumor invasion and metastasis (Gligorijevic et al., 2012; Yamaguchi, 2012; Yamaguchi et al., 2005b). Specifically, invadopodia may contribute to cancer cell invasion into the surrounding stroma, intravasation into the vasculature and extravasation (Gligorijevic et al., 2012; Paz et al., 2014). These findings collectively raised the possibility that AMFR may play a critical role in CSC-mediated cancer metastasis and thus designed a series of in vivo studies to address this possibility. To address this possibility, we transduced pancreatic cancer PANC-1 cells with a lentivirus vector encoding a fusion construct of green fluorescence protein and firefly luciferase (FFLuc; UBC-EGFP-T2A-Luc; System Biosciences, CA, USA) and GFP-positive cells were enriched by FACS. We then isolated three subpopulations of cells from the resultant PANC-1-FFLuc cells according to the expression of CD44, CD133 and AMFR, including CD133⁺CD44⁺AMFR⁺ cells (representing AMFR-positive CSCs) and CD133⁺CD44⁺AMFR⁻ cells (representing AMFR-negative CSCs). The freshly sorted cells (10⁴ cells) were then inoculated into the pancreatic tails of 8-week-old NOD/SCID mice and the tumor mass was serially quantified by bioluminescence (BLI; the IVIS Imaging System, Caliper Life Sciences, Waltham, Mass.) according to the manufacturer's recommendations. We then compared the speed and the extent of lymph node and intra-abdominal metastasis following orthotopic cell inoculation among different cell groups.

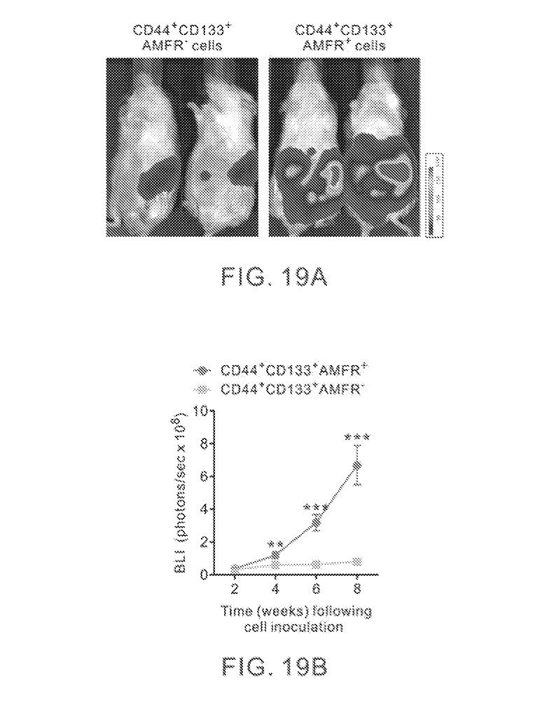

As shown in FIG. 19A-19B, continuing monitoring of the tumor masses over time revealed that CD133+CD44+AMFR+ PANC-1 CSCs grew and disseminated much faster, generating more lymph node, liver and intra-abdominal metastatic lesions than the CD133+CD44+AMFR-CSCs. This in vivo study strongly supports that AMFR-positive CSCs are more proficient in initiating primary and metastatic tumors than their AMFR-negative counterparts.

Sequence Listing

| | |
|---|---|
| <110> | Kun-Chih TSAI and Patrick Yumin YANG |
| <120> | Method for detecting and isolating invasive cancer stem cells |
| | employing cell-surface AMFR and the use thereof |
| <150> | US 62/355,362 |
| <151> | 2016-06-28 |
| <160> | 3 |
| <210> | 1 |
| <211> | 1932 |
| <212> | DNA |
| <213> | Homo sapiens |
| <400> | 1 |

| | |
|---|---|
| atgccgctgc tcttcctcga gcgcttcccc tggcccagcc tccgcaccta cacgggcctc | 60 |
| agcggcctgg ccctgctggg caccatcatc agcgcctacc gcgcgctcag ccagcccgag | 120 |
| gccggccccg gcgagccgga ccagctaacg gcctcgctgc agcctgagcc gccggcgccc | 180 |
| gcccggccga gcgccggggg accccgggcc cgcgatgtgg cccagtacct gctctcagac | 240 |
| agcctcttcg tgtgggttct agtaaatacc gcttgctgtg ttttgatgtt ggtggctaag | 300 |
| ctcatccagt gtattgtgtt tggccctctt cgagtgagtg agagacagca tctcaaagac | 360 |
| aaattttgga attttatttt ctacaagttc attttcatct ttggtgtgct gaatgtccag | 420 |
| acagtggaag aggtggtcat gtggtgcctc tggtttgccg gacttgtctt tctgcacctg | 480 |
| atggttcagc tctgcaagga tcgatttgaa tatctttcct tctcgcccac cacgccgatg | 540 |
| agcagccacg gtcgagtcct gtccctgttg gttgccatgc tgctttcctg ctgtggactg | 600 |
| gcggccgtct gctccatcac cggctacacc cacggaatgc acaccttggc tttcatggct | 660 |
| gcagagtctc ttcttgtgac agtgaggact gctcatgtga ttttacgata cgtaattcac | 720 |
| ctctgggacc tcaaccacga agggacgtgg gaaggaaagg ggacgtatgt ctattacaca | 780 |
| gactttgtca tggagctcac tctcctgtcc ctggacctca tgcaccatat tcacatgttg | 840 |
| ttatttggca acatctggtt atccatggcc agcctggtca tctttatgca gctgcgttac | 900 |
| ctgtttcatg aggtgcaacg tcgaattcgt cggcacaaga actatctacg tgtggttgga | 960 |
| aacatggagg ccaggtttgc agttgcaact ccagaggagc tggctgtcaa caatgacgac | 1020 |
| tgtgccatct gttgggactc catgcaggct gcgcggaaac tgccctgtgg acatcttttc | 1080 |
| cacaactcct gtcttcgttc ctggctagaa caagacacct cctgtccaac atgcagaatg | 1140 |
| tctcttaata ttgccgacaa taatcgtgtc agggaagaac atcaaggaga gaacttggat | 1200 |
| gagaatttgg ttcctgtagc agcagccgaa gggagacctc gcttaaacca acacaatcac | 1260 |
| ttcttccatt tcgatgggtc tcggattgcg agctggctgc cgagtttttc ggttgaagtg | 1320 |
| atgcacacca ccaacattct tggcattacg caggccagca actcccagct caatgcaatg | 1380 |
| gctcatcaga ttcaagagat gtttccccag gttccatacc atctggtact gcaggacctc | 1440 |
| cagctgacac gctcagttga aataacaaca gacaatattt tagaaggacg gattcaagta | 1500 |
| ccttttccta cacagcggtc agatagcatc agacctgcat tgaacagtcc tgtggaaagg | 1560 |
| ccaagcagtg accaggaaga gggagaaact tctgctcaga ccgagcgtgt gccactggac | 1620 |
| ctcagtcctc gcctggagga gacgctggac ttcggcgagg tggaagtgga gcccagtgag | 1680 |
| gtggaagact tcgaggctcg tgggagccgc ttctccaagt ctgctgatga gagacagcgc | 1740 |
| atgctggtgc agcgtaagga cgaactcctc cagcaagctc gcaaacgttt cttgaacaaa | 1800 |
| agttctgaag atgatgcggc ctcagagagc ttcctcccct cggaaggtgc gtcctctgac | 1860 |
| cccgtgaccc tgcgtcgaag gatgctggct gccgccgcgg aacggaggct tcagaagcag | 1920 |
| cagacctcct ag | 1932 |

| | |
|---|---|
| <210> | 2 |
| <211> | 924 |
| <212> | DNA |
| <213> | Homo sapiens |
| <400> | 2 |

| | |
|---|---|
| atgccgctgc tcttcctcga gcgcttcccc tggcccagcc tccgcaccta cacgggcctc | 60 |
| agcggcctgg ccctgctggg caccatcatc agcgcctacc gcgcgctcag ccagcccgag | 120 |
| gccggccccg gcgagccgga ccagctaacg gcctcgctgc agcctgagcc gccggcgccc | 180 |
| gcccggccga gcgccggggg accccgggcc cgcgatgtgg cccagtacct gctctcagac | 240 |
| agcctcttcg tgtgggttct agtaaatacc gcttgctgtg ttttgatgtt ggtggctaag | 300 |
| ctcatccagt gtattgtgtt tggccctctt cgagtgagtg agagacagca tctcaaagac | 360 |
| aaattttgga attttatttt ctacaagttc attttcatct ttggtgtgct gaatgtccag | 420 |
| acagtggaag aggtggtcat gtggtgcctc tggtttgccg gacttgtctt tctgcacctg | 480 |
| atggttcagc tctgcaagga tcgatttgaa tatctttcct tctcgcccac cacgccgatg | 540 |
| agcagccacg gtcgagtcct gtccctgttg gttgccatgc tgctttcctg ctgtggactg | 600 |
| gcggccgtct gctccatcac cggctacacc cacggaatgc acaccttggc tttcatggct | 660 |
| gcagagtctc ttcttgtgac agtgaggact gctcatgtga ttttacgata cgtaattcac | 720 |
| ctctgggacc tcaaccacga agggacgtgg gaaggaaagg ggacgtatgt ctattacaca | 780 |
| gactttgtca tggagctcac tctcctgtcc ctggacctca tgcaccatat tcacatgttg | 840 |
| ttatttggca acatctggtt atccatggcc agcctggtca tctttatgca gctgcgttac | 900 |
| ctgtttcatg aggtgcaacg tcga | 924 |

| | |
|---|---|
| <210> | 3 |
| <211> | 795 |
| <212> | DNA |
| <213> | Homo sapiens |
| <400> | 3 |

| | |
|---|---|
| atgtctctta atattgccga caataatcgt gtcagggaag aacatcaagg agagaacttg | 60 |
| gatgagaatt tggttcctgt agcagcagcc gaagggagac ctcgcttaaa ccaacacaat | 120 |
| cacttcttcc atttcgatgg gtctcggatt gcgagctggc tgccgagttt ttcggttgaa | 180 |
| gtgatgcaca ccaccaacat tcttggcatt acgcaggcca gcaactccca gctcaatgca | 240 |
| atggctcatc agattcaaga gatgtttccc caggttccat accatctggt actgcaggac | 300 |
| ctccagctga cacgctcagt tgaaataaca acagacaata ttttagaagg acggattcaa | 360 |
| gtaccttttc ctacacagcg gtcagatagc atcagacctg cattgaacag tcctgtggaa | 420 |
| aggccaagca gtgaccagga agagggagaa acttctgctc agaccgagcg tgtgccactg | 480 |
| gacctcagtc ctcgcctgga ggagacgctg gacttcggcg aggtggaagt ggagcccagt | 540 |
| gaggtggaag acttcgaggc tcgtgggagc cgcttctcca agtctgctga tgagagacag | 600 |
| cgcatgctgg tgcagcgtaa ggacgaactc ctccagcaag ctcgcaaacg tttcttgaac | 660 |
| aaaagttctg aagatgatgc ggcctcagag agcttcctcc cctcggaagg tgcgtcctct | 720 |
| gaccccgtga ccctgcgtcg aaggatgctg gctgccgccg cggaacggag gcttcagaag | 780 |
| cagcagacct cctag | 795 |

1.-26. (canceled)

27. An isolated invasive cancer stem cell (iCSC) or a substantially homogeneous iCSC population comprising said iCSC, which is positive for the marker AMFR on the cell membrane.

28. The iCSC of claim 27, which is characterized by: (a) having stem-cell like properties; and (b) having invasive properties.

29. The iCSC of claim 27, wherein said stem-cell-like properties include: (a) expressing stem cell markers, which comprise, CD133, CD44, CD24, CD90, CD15, CD20, CD117, CD166, CD271, epithelial specific antigen (ESA), CXCR4, aldehyde dehydrogenase (ALDH), c-Met, nestin, nodal-activin, ABCG2, alpha2betal-integrin, alpha6-integrin or any combination of the foregoing; (b) expressing a low level of CD24 if said solid tumor is a breast cancer or a prostate cancer; (c) giving rise to additional stem-cell-like tumor cells; (d) being able to form a detectable tumor upon transplantation into an immunocompromised host; and/or (e) being able to regenerate the hierarchical organization of solid tumor tissues.

30. The iCSC of claim 27, wherein said cell membrane is within, the regions of invadopodia-like structures, said invadopodia-like structures are transient actin-based protrusions on a cancer cell that mediate focal degradation of extracellular matrix and cell invasion and tumor metastasis.

31. A method of detecting an isolated invasive cancer stem cell (iCSC) which is positive for the marker AMFR on the cell membrane within an established cancer or a collection of cancer cells, wherein the method comprises: (a) preparing a sample of said cancer or said collection of cancer cells; (b) contacting said sample with an agent that binds to AMFR on the membrane; and (c) determining whether said sample contains cancer cells expressing AMFR on the cell membrane, regardless of the expression of AMFR in the cell cytosol or nuclei, thereby detecting said iCSC.

32. The method of claim 31, wherein said cancer comprises, pancreatic, lung, prostate, liver, gastric, oral, esophageal, breast, kidney, bladder cancers, as well as neuroendocrine tumors, cholangiocarcinoma, malignant glioma, lymphoma, acute myeloid leukemia, chronic myeloid leukemia, acute lymphocytic leukemia or chronic lymphocytic leukemia.

33. The method of claim 31, wherein said sample is a biopsy specimen, a surgical specimen, a blood-derived specimen, a urine specimen, a stool specimen, a cerebral spinal fluid specimen, a biliary juice specimen, a pancreatic juice specimen, cultured cells, and/or any combination thereof.

34. The method of claims 31, wherein said agent is selected from a group consisting of an antibody or the like, a peptide, an aptamer, or any molecule or compound that is sufficient to confer specific binding to AMFR on the cell surface with high affinity.

35. The method of claim 34, said antibody includes: polyclonal, monoclonal, genetically engineered forms of antibodies, chimeric antibodies, humanized antibodies, hetero-conjugate antibodies, single chain variable fragment (scFv) antibodies, polypeptides that contain at least a portion of an immunoglobulin that is sufficient to confer specific antigen biding to the polypeptide, or antigen binding fragments of antibodies.

36. The method of claim 31, wherein said determining whether said sample contains cancer cells that express AMFR on the cell membrane comprise the methods of, immunofluorescence staining, immunohistochemistry, in situ PCR, ELISA, immunoblotting, proximity ligation analysis, flow cytometry, mass spectrometry, or protein, tissue or cell microarray.

37. A method of isolating an isolated invasive cancer stem cell (iCSC) which is positive for the marker AMFR on the cell membrane or a substantially homogeneous cell population comprising said iCSC from an established cancer or a collection of cancer cells, wherein the method comprises: (a) preparing a sample of said cancer or said collection of cancer cells; (b) contacting said sample with a binding agent that binds to AMFR on the cell surface; and (c) isolating cancer cells from said sample that express AMFR on the cell membrane, thereby isolating said iCSC.

38. The method of claim 37, wherein said method of isolating said iCSC is selected from the group consisting of fluorescence-activated cell sorting, magnetic-assisted cell sorting, or any means that are capable of selecting cells based on specific protein epitopes present on the cell surface.


SEQUENCE LISTING (3 sequences)

SEQ ID NO: 1 - DNA, 1932 bp, Homo sapiens
atgccgctgctcttcctcgagcgcttcccctggcccagcctccgcacctacacgggcctcagcggcctggccctgctgggcaccatcatcagcgcctaccgcgcgctcagccagcccgaggccggccccggcgagccggaccagctaacggcctcgctgcagcctgagccgccggcgcccgcccggccgagcgccgggggaccccgggcccgcgatgtggcccagtacctgctctcagacagcctcttcgtgtgggttctagtaaataccgcttgctgtgttttgatgttggtggctaagctcatccagtgtattgtgtttggccctcttcgagtgagtgagagacagcatctcaaagacaaattttggaattttattttctacaagttcattttcatctttggtgtgctgaatgtccagacagtggaagaggtggtcatgtggtgcctctggtttgccggacttgtctttctgcacctgatggttcagctctgcaaggatcgatttgaatatctttccttctcgcccaccacgccgatgagcagccacggtcgagtcctgtccctgttggttgccatgctgctttcctgctgtggactggcggccgtctgctccatcaccggctacacccacggaatgcacaccttggctttcatggctgcagagtctcttcttgtgacagtgaggactgctcatgtgattttacgatacgtaattcacctctgggacctcaaccacgaagggacgtgggaaggaaaggggacgtatgtctattacacagactttgtcatggagctcactctcctgtccctggacctcatgcaccatattcacatgttgttatttggcaacatctggttatccatggccagcctggtcatctttatgcagctgcgttacctgtttcatgaggtgcaacgtcgaattcgtcggcacaagaactatctacgtgtggttggaaacatggaggccaggtttgcagttgcaactccagaggagctggctgtcaacaatgacgactgtgccatctgttgggactccatgcaggctgcgcggaaactgccctgtggacatcttttccacaactcctgtcttcgttcctggctagaacaagacacctcctgtccaacatgcagaatgtctcttaatattgccgacaataatcgtgtcagggaagaacatcaaggagagaacttggatgagaatttggttcctgtagcagcagccgaagggagacctcgcttaaaccaacacaatcacttcttccatttcgatgggtctcggattgcgagctggctgccgagtttttcggttgaagtgatgcacaccaccaacattcttggcattacgcaggccagcaactcccagctcaatgcaatggctcatcagattcaagagatgtttccccaggttccataccatctggtactgcaggacctccagctgacacgctcagttgaaataacaacagacaatattttagaaggacggattcaagtaccttttcctacacagcggtcagatagcatcagacctgcattgaacagtcctgtggaaaggccaagcagtgaccaggaagagggagaaacttctgctcagaccgagcgtgtgccactggacctcagtcctcgcctggaggagacgctggacttcggcgaggtggaagtggagcccagtgaggtggaagacttcgaggctcgtgggagccgcttctccaagtctgctgatgagagacagcgcatgctggtgcagcgtaaggacgaactcctccagcaagctcgcaaacgtttcttgaacaaaagttctgaagatgatgcggcctcagagagcttcctcccctcggaaggtgcgtcctctgaccccgtgaccctgcgtcgaaggatgctggctgccgccgcggaacggaggcttcagaagcagcagacctcctag

SEQ ID NO: 2 - DNA, 924 bp, Homo sapiens
atgccgctgctcttcctcgagcgcttcccctggcccagcctccgcacctacacgggcctcagcggcctggccctgctgggcaccatcatcagcgcctaccgcgcgctcagccagcccgaggccggccccggcgagccggaccagctaacggcctcgctgcagcctgagccgccggcgcccgcccggccgagcgccgggggaccccgggcccgcgatgtggcccagtacctgctctcagacagcctcttcgtgtgggttctagtaaataccgcttgctgtgttttgatgttggtggctaagctcatccagtgtattgtgtttggccctcttcgagtgagtgagagacagcatctcaaagacaaattttggaattttattttctacaagttcattttcatctttggtgtgctgaatgtccagacagtggaagaggtggtcatgtggtgcctctggtttgccggacttgtctttctgcacctgatggttcagctctgcaaggatcgatttgaatatctttccttctcgcccaccacgccgatgagcagccacggtcgagtcctgtccctgttggttgccatgctgctttcctgctgtggactggcggccgtctgctccatcaccggctacacccacggaatgcacaccttggctttcatggctgcagagtctcttcttgtgacagtgaggactgctcatgtgattttacgatacgtaattcacctctgggacctcaaccacgaagggacgtgggaaggaaaggggacgtatgtctattacacagactttgtcatggagctcactctcctgtccctggacctcatgcaccatattcacatgttgttatttggcaacatctggttatccatggccagcctggtcatctttatgcagctgcgttacctgtttcatgaggtgcaacgtcga

SEQ ID NO: 3 - DNA, 795 bp, Homo sapiens
atgtctcttaatattgccgacaataatcgtgtcagggaagaacatcaaggagagaacttggatgagaatttggttcctgtagcagcagccgaagggagacctcgcttaaaccaacacaatcacttcttccatttcgatgggtctcggattgcgagctggctgccgagtttttcggttgaagtgatgcacaccaccaacattcttggcattacgcaggccagcaactcccagctcaatgcaatggctcatcagattcaagagatgtttccccaggttccataccatctggtactgcaggacctccagctgacacgctcagttgaaataacaacagacaatattttagaaggacggattcaagtaccttttcctacacagcggtcagatagcatcagacctgcattgaacagtcctgtggaaaggccaagcagtgaccaggaagagggagaaacttctgctcagaccgagcgtgtgccactggacctcagtcctcgcctggaggagacgctggacttcggcgaggtggaagtggagcccagtgaggtggaagacttcgaggctcgtgggagccgcttctccaagtctgctgatgagagacagcgcatgctggtgcagcgtaaggacgaactcctccagcaagctcgcaaacgtttcttgaacaaaagttctgaagatgatgcggcctcagagagcttcctcccctcggaaggtgcgtcctctgaccccgtgaccctgcgtcgaaggatgctggctgccgccgcggaacggaggcttcagaagcagcagacctcctag